(19) United States
(12) Patent Application Publication
(10) Pub. No.: US 2021/0069362 A1
(43) Pub. Date: Mar. 11, 2021

(54) DEVICE DISINFECTOR

(71) Applicant: SOCLEAN, INC., PETERBOROUGH, NH (US)

(72) Inventors: Michelle Gregory, Greenfield, NH (US); Matthew McCambridge, Lexington, MA (US); Julianna Miller, Boston, MA (US); Mike Le, Somerville, MA (US); Kurt MAW, Salem, MA (US); Ashley James Nye LEGG, Newton, MA (US); Robert WILKINS, Peterborough, NH (US); Robert A. CHARLES, New Boston, NH (US); Alex CHAVES, Hudson, NH (US); Gregory Randall LANIER, JR., Merrimack, NH (US)

(73) Assignee: SOCLEAN, INC., PETERBOROUGH, NH (US)

(21) Appl. No.: 17/013,198

(22) Filed: Sep. 4, 2020

Related U.S. Application Data
(63) Continuation-in-part of application No. PCT/US20/23631, filed on Mar. 19, 2020.
(60) Provisional application No. 62/979,551, filed on Feb. 21, 2020.
(60) Provisional application No. 62/896,117, filed on Sep. 5, 2019.
(60) Provisional application No. 63/010,297, filed on Apr. 15, 2020.
(60) Provisional application No. 63/038,573, filed on Jun. 12, 2020.

(51) Int. Cl. A61L 2/20

(52) U.S. Cl. CPC A61L 2/202; A61L 2202/15; A61L 2202/122; A61L 2202/11

(57) ABSTRACT

A sanitization device may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter access door transitionable between an open position and a closed position, the filter access door including a filter holder configured to receive a filter such that the filter fluidly couples to the ozone outlet when the filter access door is in the closed position, the filter is configured to reduce ozone passing therethrough to oxygen and the filter is downstream of the connector, and an exhaust fan fluidly coupled to the ozone outlet and configured to urge ozone through the filter.

### CROSS-REFERENCE TO RELATED APPLICATIONS

The present application is a continuation in part of International Application No. PCT/US20/23631 filed on Mar. 19, 2020, and entitled Technologies for Sanitizing Medical Devices, which claims the priority to U.S. Provisional Application Ser. No. 62/979,551 filed on Feb. 21, 2020, entitled Technologies for Sanitizing Medical Devices, U.S. Provisional Application Ser. No. 62/896,117 filed on Sep. 5, 2019, entitled Technologies for Sanitizing Medical Devices, and U.S. Provisional Application Ser. No. 62/820,624 filed on Mar. 19, 2019, entitled Technologies for Sanitizing Medical Devices, and the present application claims priority to U.S. Provisional Application Ser. No. 63/010,297 filed on Apr. 15, 2020, entitled Sanitization Device and Adaptor for use with the same, U.S. Provisional Application Ser. No. 63/038,573 filed on Jun. 12, 2020, entitled Sanitization Device, U.S. Provisional Application Ser. No. 62/979,551 filed on Feb. 21, 2020, entitled Technologies for Sanitizing Medical Devices, and U.S. Provisional Application Ser. No. 62/896,117 filed on Sep. 5, 2019, entitled Technologies for Sanitizing Medical Devices, each of which are incorporated herein by reference.

### TECHNICAL FIELD

The present disclosure is generally directed to sanitization devices (which may also be referred to as device disinfectors) and more specifically to a sanitization device configured to sanitize (e.g., disinfect) an article using a sanitization fluid (e.g., ozone gas) that is incident on one or more surfaces of the device.

### BACKGROUND INFORMATION

Personal articles (e.g., keys, wallets, jewelry, eyeglasses, electronics, and/or any other personal articles) are exposed to numerous environments in which harmful bacteria and/or viruses can come into contact with and reside on the articles. Without proper sanitization, further use of these articles after exposure can result in the spread of harmful bacteria and/or viruses. Accordingly, before reuse, articles may be sanitized in order to reduce the risk of spreading harmful bacteria and viruses.

### BRIEF DESCRIPTION OF THE DRAWINGS

These and other features advantages will be better understood by reading the following detailed description, taken together with the drawings wherein:

FIG. 1 shows a schematic block diagram of an example of a sanitization device, consistent with embodiments of the present disclosure.

FIG. 2 shows a schematic cross-sectional view of an example of a sanitization device, consistent with embodiments of the present disclosure.

FIG. 3 shows a schematic cross-sectional view of an example of the sanitization device of FIG. 2 taken along the line III-III, wherein the example sanitization device has a first example configuration of a filter and an exhaust fan, consistent with embodiments of the present disclosure.

FIG. 4 shows a schematic cross-sectional view of an example of the sanitization device of FIG. 2 taken along the line III-III, wherein the example sanitization device has a second example configuration of the filter and the exhaust fan, consistent with embodiments of the present disclosure.

FIG. 5 shows a schematic cross-sectional view of an example of the sanitization device of FIG. 2 taken along the line III-III, wherein the example sanitization device has a third example configuration of the filter and the exhaust fan, consistent with embodiments of the present disclosure.

FIG. 6 shows a schematic cross-sectional view of an example of the sanitization device of FIG. 2 taken along the line III-III, wherein the example sanitization device has a fourth example configuration of the filter and the exhaust fan, consistent with embodiments of the present disclosure.

FIG. 7 shows a schematic cross-sectional view of an example of the sanitization device of FIG. 2 taken along the line III-III, wherein the example sanitization device has a fifth example configuration of the filter and the exhaust fan, consistent with embodiments of the present disclosure.

FIG. 8 shows a schematic cross-sectional view of an example of the sanitization device of FIG. 2 taken along the line III-III, wherein the example sanitization device has a sixth example configuration of the filter and the exhaust fan, consistent with embodiments of the present disclosure.

FIG. 9 shows a schematic cross-sectional view of an example of the sanitization device of FIG. 2 taken along the line III-III, wherein the example sanitization device has a seventh example configuration of the filter and the exhaust fan, consistent with embodiments of the present disclosure.

FIG. 10 shows a schematic cross-sectional view of an example of the sanitization device of FIG. 2 taken along the line III-III, wherein the example sanitization device has an eighth example configuration of the filter and the exhaust fan, consistent with embodiments of the present disclosure.

FIG. 11 shows schematic block diagram of an ozone operating system of the sanitization device of FIG. 2, consistent with embodiments of the present disclosure.

FIG. 12 shows a schematic block diagram of a controller, which may control and/or monitor one or more operations of the sanitization device of FIG. 2, consistent with embodiments of the present disclosure.

FIG. 13 shows a front perspective view of a sanitization device, consistent with embodiments of the present disclosure.

FIG. 14 shows a rear perspective view of the sanitization device of FIG. 13, consistent with embodiments of the present disclosure.

FIG. 15 shows a cross-sectional view of the sanitization device of FIG. 13 taken along the line XV-XV, consistent with embodiments of the present disclosure.

FIG. 16 shows a magnified view generally corresponding to the region XVI of FIG. 15, consistent with embodiments of the present disclosure.

FIG. 17 shows a cross-sectional view of the sanitization device of FIG. 13 taken along the line XVII-XVII, consistent with embodiments of the present disclosure.

FIG. 18 shows a perspective view of a filter of the sanitization device of FIG. 13, consistent with embodiments of the present disclosure.

FIG. 19 shows a cross-sectional view of the filter of FIG. 18 taken along the line XIX-XIX, consistent with embodiments of the present disclosure.

FIG. 20 shows a schematic perspective view of an example of a sanitization accessory, consistent with embodiments of the present disclosure.

FIG. 21 shows a schematic perspective view of an example of a sanitization accessory, consistent with embodiments of the present disclosure.

FIG. 22 shows a schematic perspective view of an example of a sanitization accessory, consistent with embodiments of the present disclosure.

FIG. 23 shows a schematic perspective view of an example of a sanitization accessory, consistent with embodiments of the present disclosure.

FIG. 24 shows a schematic perspective view of an example of a sanitization accessory, consistent with embodiments of the present disclosure.

FIG. 25 shows a schematic perspective view of an example of a sanitization accessory, consistent with embodiments of the present disclosure.

FIG. 26 shows a cross-sectional view of the sanitization accessory of FIG. 25 taken along the line XXVI-XXVI, wherein the sanitization accessory of FIG. 24 is disposed within the sanitization accessory of FIG. 25, consistent with embodiments of the present disclosure.

FIG. 27 shows a cross-sectional view of a sanitization device having the sanitization accessories of FIGS. 24 and 25 disposed therein, consistent with embodiments of the present disclosure.

FIG. 28 shows a perspective view of a modular sanitization accessory, consistent with embodiments of the present disclosure.

FIG. 29 shows a cross-sectional perspective view of a portion of a sanitization compartment having the modular sanitization accessory of FIG. 28 disposed therein, consistent with embodiments of the present disclosure.

FIG. 30 shows another cross-sectional perspective view of a portion of the sanitization compartment of FIG. 29 having the modular sanitization accessory of FIG. 28 and a basket disposed therein, consistent with embodiments of the present disclosure.

FIG. 31 shows a perspective view of the basket of FIG. 30, consistent with embodiments of the present disclosure.

FIG. 32 shows another cross-sectional perspective view of a portion of the sanitization compartment of FIG. 29 having the modular sanitization accessory of FIG. 28 and a plurality of articles to be sanitized disposed therein, consistent with embodiments of the present disclosure.

FIG. 33 shows a perspective view of an example of a sanitization accessory, consistent with embodiments of the present disclosure.

FIG. 34 shows a perspective top view of a portion of a sanitization compartment having the sanitization accessory of FIG. 33 disposed therein, consistent with embodiments of the present disclosure.

FIG. 35 shows a perspective view of an example of a sanitization accessory, consistent with embodiments of the present disclosure.

FIG. 36 shows another perspective view of the sanitization accessory of FIG. 35, consistent with embodiments of the present disclosure.

FIG. 37 shows a bottom perspective view of the sanitization accessory of FIG. 35, consistent with embodiments of the present disclosure.

FIG. 38 shows a perspective view of protrusions and accessory feet extending from a substrate that is configured to be coupled to a platform of the sanitization accessory of FIG. 35, consistent with embodiments of the present disclosure.

FIG. 39 shows a perspective view of an accessory rack of the sanitization accessory of FIG. 35, consistent with embodiments of the present disclosure.

FIG. 40 shows a cutout configured to be bent into the shape of the accessory rack of FIG. 39, consistent with embodiments of the present disclosure.

FIG. 41 shows a perspective view of an example of a sanitization device, consistent with embodiments of the present disclosure.

FIG. 42 shows a cross-sectional perspective view of the sanitization device of FIG. 41 taken along the line XLII-XLII, consistent with embodiments of the present disclosure.

FIG. 43 shows another perspective view of the sanitization device of FIG. 41, wherein a filter access door is in an open position, consistent with embodiments of the present disclosure.

FIG. 44 shows a perspective view of an example of a sanitization device, consistent with embodiments of the present disclosure.

FIG. 45 shows another perspective view of the sanitization device of FIG. 44, wherein a filter access door is in an open position, consistent with embodiments of the present disclosure.

FIG. 46 shows a cross-sectional view of the sanitization device of FIG. 44 generally corresponding to the region XLVI of FIG. 44, consistent with embodiments of the present disclosure.

FIG. 47 shows a cross-sectional view of the sanitization device of FIG. 45 generally corresponding to region XLVII of FIG. 45, consistent with embodiments of the present disclosure.

FIG. 48 shows a perspective view of a sanitization accessory having a first component and a second component, consistent with embodiments of the present disclosure.

FIG. 49 shows a perspective exploded view of the sanitization accessory of FIG. 48, consistent with embodiments of the present disclosure.

FIG. 50 shows a bottom perspective view of the first component of the sanitization accessory of FIG. 48, consistent with embodiments of the present disclosure.

FIG. 51 shows a top perspective view of the first component of the sanitization accessory of FIG. 48, consistent with embodiments of the present disclosure.

### DETAILED DESCRIPTION

The present disclosure is generally directed to a sanitization device configured to sanitize (e.g., disinfect) one or more personal articles (e.g., keys, wallets, jewelry, eyeglasses, electronics, and/or any other personal articles). The sanitization device includes a sanitization compartment configured to receive the personal article, a sanitizing fluid operating system configured to generate a sanitizing fluid (e.g., ozone gas), a distribution line to deliver the sanitizing fluid to the sanitization compartment, and an exhaust fan downstream of the sanitizing fluid operating system that is configured to urge the sanitizing fluid through a filter when the sanitizing fluid operating system is generating the sanitizing fluid.

As used herein, the term “fan” may generally refer to any device capable of moving a fluid. For example, an exhaust fan may be a fan, a pump, a compressor, or a blower. By way of further example, a distribution fan may be a fan, a pump, a compressor, or a blower.

Although the technologies described herein can be used with many sanitizing fluids, the present disclosure focuses on the use of ozone as a sanitizing gas. This is because ozone (03) gas is an effective sanitizer yet is relatively safe for consumer use. Because of its strong oxidizing properties, ozone can effectively kill or otherwise remove a wide range of organic and inorganic contaminants such as yeasts, bacteria, molds, viruses, other pathogens, and/or pollutants with which it comes into contact, e.g., via oxidation. Naturally over time and/or as it oxidizes contaminants, ozone is chemically reduced to oxygen (O₂), which is safe for human consumption and for release into the environment. Ozone is also relatively easy to generate on site (and thus does not require the use of a storage tank) and leaves little or no chemical residue. For those and other reasons, ozone has been identified as a safe and effective sanitizing gas for use in the present disclosure. It should be understood, however, that the technologies described herein are not limited to the use of ozone and may be employed with a wide variety of sanitizing fluids.

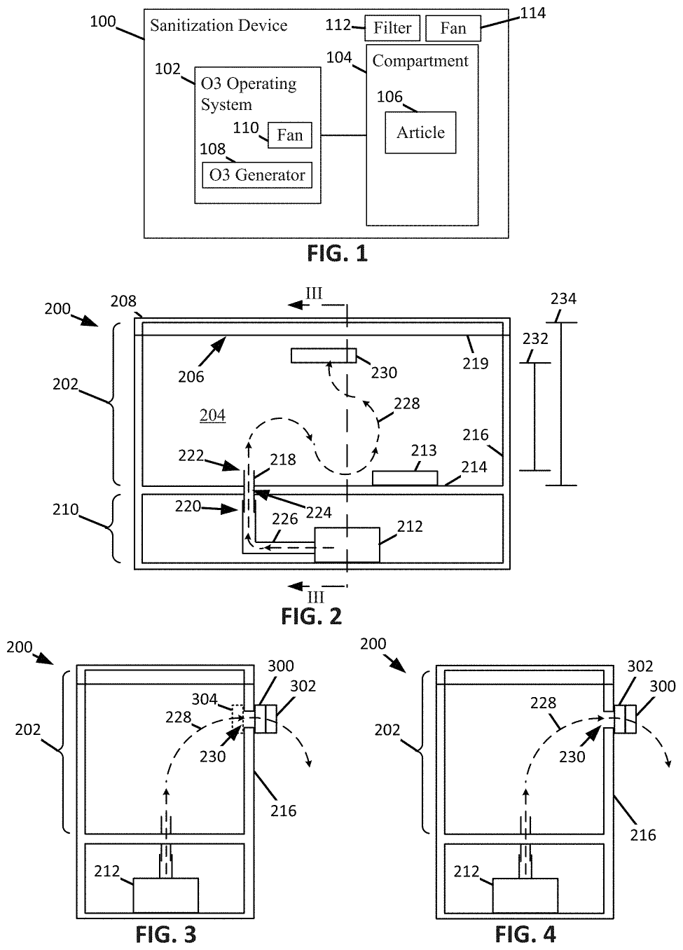

FIG. 1 shows a schematic block diagram of a sanitization device 100. As shown, the sanitization device 100 includes an ozone operating system 102 and a sanitization compartment 104. The ozone operating system 102 is configured to generate ozone and is fluidly coupled to the sanitization compartment 104. The sanitization compartment 104 is configured to receive at least one personal article to be sanitized 106 (e.g., keys, wallets, jewelry, eyeglasses, electronics, and/or any other personal articles) therein. In operation, ozone is generated by the ozone operating system 102 and caused to flow into the sanitization compartment 104. Once in the sanitization compartment 104, the ozone comes into contact with the article 106, sanitizing the article 106. Before being exhausted from the sanitization compartment 104, the ozone may be caused to be broken down into oxygen (e.g., by passing the ozone through a filter 112).

In some instances, the ozone operating system 102 includes an ozone generator 108 and a distribution fan 110. The ozone generator 108 generates ozone and the distribution fan 110 urges the ozone into the sanitization compartment 104. In some instances, the ozone operating system 102 may be external to the sanitization compartment 104. In this instance, the ozone operating system 102 may be fluidly coupled to the sanitization compartment 104 through an ozone distribution line. In other instances, the ozone operating system 102 is disposed within the sanitization compartment 104 such that ozone generated is dispersed directly into the sanitization compartment 104 from the ozone operating system 102.

The sanitization device 100 may include at least one exhaust fan 114 positioned downstream of the ozone operating system 102. The exhaust fan 114 is fluidly coupled to the sanitization compartment 104 and configured to urge ozone within the sanitization compartment 104 to pass through the filter 112. The exhaust fan 114 can be disposed within the sanitization compartment 104 or external to the sanitization compartment 104. For example, the exhaust fan 114 can be disposed along an ozone flow path extending within the sanitization device 100 at a location between the ozone operating system 102 and the filter 112. In other words, the exhaust fan 114 can be disposed upstream of the ozone operating system 102 and downstream of filter 112. By way of further example, the filter 112 can be disposed along the ozone flow path at a location between the ozone operating system 102 and the exhaust fan 114. In other words, the exhaust fan 114 can be disposed downstream of both the ozone operating system 102 and the filter 112. In some instances, the sanitization device 100 may include a plurality of exhaust fans 114, wherein at least one exhaust fan 114 is upstream of the filter 112 and at least one exhaust fan 114 is downstream of the filter 112 (see, e.g., FIG. 3).

The exhaust fan 114 may be fluidly coupled to an outlet of the sanitization compartment 104 and the ozone operating system 102 may be fluidly coupled to an inlet of the sanitization compartment 104. In some instances, the outlet and the inlet may be defined in the same sidewall of the sanitization compartment 104 (e.g., a bottom of the sanitization compartment 104). In other instances, the outlet and the inlet may be defined in different sidewalls of the sanitization compartment 104 (e.g., the inlet may be defined in a bottom of the sanitization compartment 104 and the outlet may be defined in a sidewall extending from the bottom of the sanitization compartment 104). When the inlet is defined in a bottom of the sanitization compartment 104, the outlet may be defined in a sidewall extending from the bottom at a location proximate to (e.g., spaced apart by a distance measuring less than or equal to 5%, 10%, or 15% of a total height of the sanitization compartment) the bottom. Alternatively, when the inlet is defined in a bottom of the sanitization compartment 104, the outlet may be defined in a sidewall extending from the bottom at a location proximate to (e.g., spaced apart by a distance measuring less than or equal to 5%, 10%, or 15% of a total height of the sanitization compartment) an open end and/or lid of the sanitization compartment 104.

FIG. 2 shows a schematic cross-sectional view of a sanitization device 200, which may be an example of the sanitization device 100 of FIG. 1. The sanitization device 200 is shown without an exhaust fan or filter coupled thereto for purposes of clarity. Example embodiments of the sanitization device 200 showing various configurations of a filter and an exhaust fan are discussed later herein.

As shown, the sanitization device 200 includes a sanitization compartment 202 defining a sanitization cavity 204 having at least one open end 206 and a lid 208 extending over the open end 206 and configured to selectively enclose the sanitization cavity 204. The sanitization device 200 further includes a hardware compartment 210 for receiving an ozone operating system 212 configured to generate ozone gas to be delivered to the sanitization compartment 202 such that an article 213 within the sanitization compartment 202 can be sanitized.

As also shown, the sanitization compartment 202 includes a bottom (or base) 214 and one more sidewalls 216 that extend from the bottom 214 in direction of the lid 208. When the lid 208 is in a closed position, the lid 208 sealingly engages with the one or more sidewalls 216. For example, a continuous seal may be formed at an interface 219 between the lid 208 and the one or more sidewalls 216. The bottom 214 extends between the lid 208 and the hardware compartment 210. As such, the bottom 214 separates the sanitization compartment 202 from the hardware compartment 210.

A connector 218 having a first end 220 and a second end 222 extends through the bottom 214 such that the first end 220 is disposed within the hardware compartment 210 and the second end 222 is disposed within the sanitization compartment 202. The connector 218 defines a fluid pathway 224 that extends between the first and second ends 220 and 222. As such, the connector 218 may generally be described as defining an inlet to the sanitization compartment 202.

The first end 220 of the connector 218 is received within an ozone distribution line 226 such that the ozone distribution line 226 is fluidly coupled to the fluid pathway 224 of the connector 218. The ozone distribution line 226 is further fluidly coupled to the ozone operating system 212. As shown, an ozone flow path 228 extends from the ozone operating system 212 through the ozone distribution line 226 and the fluid pathway 224 and into the sanitization compartment 202. From the connector 218, the ozone flow path 228 extends through the sanitization compartment 202 (such that ozone can be distributed throughout the sanitization compartment 202) and into an ozone (or compartment) outlet 230 defined in at least one of the one or more sidewalls 216 such that ozone flowing along the ozone flow path 228 is exhausted from the sanitization cavity 204. Before being exhausted into a surrounding environment, at least a portion of ozone flowing along the ozone flow path 228 can be reduced to oxygen (e.g., using a filter).

As shown, the ozone outlet 230 is vertically spaced apart from the second end 222 of the connector 218 by a vertical separation distance 232. A ratio of the vertical separation distance 232 to a sanitization compartment height 234 (i.e., the vertical separation distance 232 divided by the sanitization compartment height 234) may be, for example, greater than 0.25. By way of further example, the ratio of the vertical separation distance 232 to the sanitization compartment height 234 may be greater than 0.5. By way of still further example, the ratio of the vertical separation distance 232 to the sanitization compartment height 234 may be greater than 0.75. By way of still further example, the ratio of the vertical separation distance 232 to the sanitization compartment height 234 may be greater than 0.85. By way of still further example, the ratio of the vertical separation distance 232 to the sanitization compartment height 234 may be greater than 0.95. As the ratio of the vertical separation distance 232 to the sanitization compartment height 234 increases, the quantity of ozone dispersed within the sanitization compartment 202 may also increase. As such, in some instances, the vertical separation distance 232 may be maximized (e.g., the ratio of the vertical separation distance 232 to the sanitization compartment height 234 is greater than 0.7). Alternatively, in some instances, the vertical separation distance 232 may be minimized (e.g., the ratio of the vertical separation distance 232 to the sanitization compartment height 234 is less than 0.3).

While the connector 218 is shown as being in the bottom 214 and the ozone outlet 230 is shown as being in the sidewall 216, other configuration are possible. For example, the connector 218 may be in at least one of the one or more sidewalls 216 and the ozone outlet 230 may be defined in the bottom 214. In this example, the density of ozone, relative to air, encourages ozone passing out of the connector 218 to move in a direction of the bottom 214, which may encourage ozone to be exhausted from the ozone outlet 230.

FIG. 3 shows a schematic cross-sectional view of the sanitization device 200 of FIG. 2 taken along the line III-III, wherein the sanitization device 200 has a first example configuration for a filter 300 and an exhaust fan 302. As shown, according to the first example configuration, the filter 300 may be disposed within the ozone flow path 228 at a location between the ozone outlet 230 and the exhaust fan 302. As shown, the filter 300 and exhaust fan 302 are external to the sanitization compartment 202, wherein the filter 300 and the exhaust fan 302 are coupled to the sidewall 216. In other words, the filter 300 and the exhaust fan 302 are disposed at location external to the sanitization compartment 202. As such, the ozone flow path 228 extends from the ozone operating system 212 and through both the ozone outlet 230 and the filter 300 before extending through the exhaust fan 302. Therefore, the exhaust fan 302 may be generally described as being downstream of the ozone operating system 212, the ozone outlet 230, and the filter 300. In this configuration, the exhaust fan 302 is configured urge ozone within the sanitization compartment 202 to flow through the filter 300 utilizing a suction force generated by the exhaust fan 302.

In some instances, the sanitization device 200 may include an optional secondary exhaust fan 304 (shown in broken lines). As shown, the optional secondary exhaust fan 304 and the exhaust fan 302 may be disposed on opposing sides of the filter 300.

FIG. 4 shows a schematic cross-sectional view of the sanitization device 200 of FIG. 2 taken along the line III-III, wherein the sanitization device 200 has a second example configuration for the filter 300 and the exhaust fan 302. As shown, according to the second example configuration, the exhaust fan 302 may be disposed within the ozone flow path 228 at a location between the ozone outlet 230 and the filter 300. The exhaust fan 302 and the filter 300 are external to the sanitization compartment 202, wherein the filter 300 and the exhaust fan 302 are coupled to the sidewall 216. In other words, the filter 300 and the exhaust fan 302 are disposed at location external to the sanitization compartment 202. As such, the ozone flow path 228 extends from the ozone operating system 212 and through both the ozone outlet 230 and the exhaust fan 302 before extending through the filter 300. Therefore, the filter 300 may be generally described as being downstream of the ozone operating system 212, the ozone outlet 230, and the exhaust fan 302. In this configuration, the exhaust fan 302 is configured urge ozone within the sanitization compartment 202 to flow through the filter 300 utilizing a blowing force generated by the exhaust fan 302.

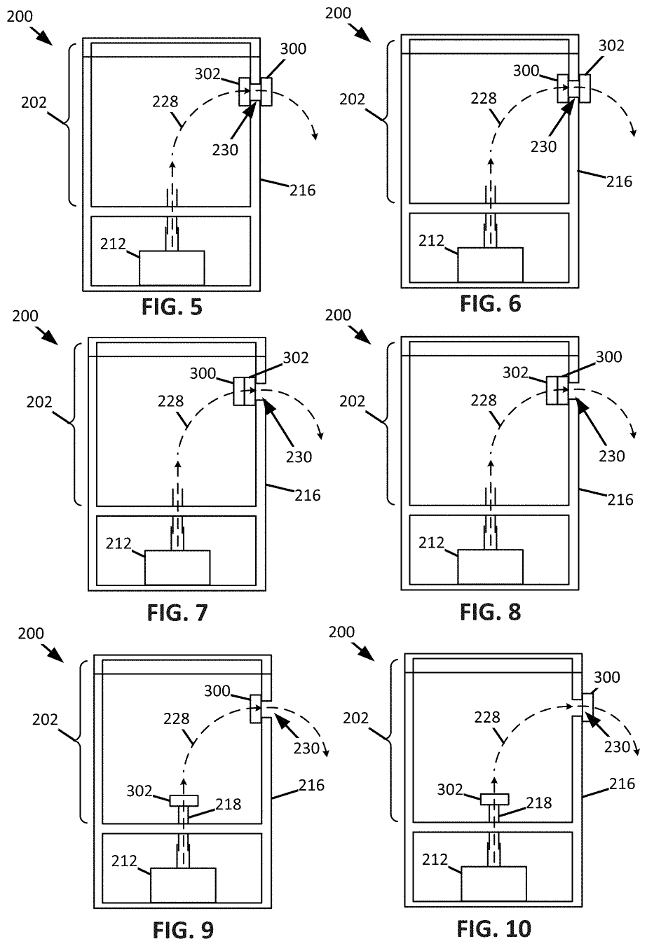

FIG. 5 shows a schematic cross-sectional view of the sanitization device 200 of FIG. 2 taken along the line III-III, wherein the sanitization device 200 has a third example configuration for the filter 300 and the exhaust fan 302. As shown, according to the third example configuration, the ozone outlet 230 may be disposed within the ozone flow path 228 at a location between the exhaust fan 302 and the filter 300, wherein the exhaust fan 302 is within the sanitization compartment 202 and the filter 300 is external to the sanitization compartment 202. The filter 300 and the exhaust fan 302 are coupled to the sidewall 216. As such, the ozone flow path 228 extends from the ozone operating system 212 and through both the exhaust fan 302 and the ozone outlet 230 before extending through the filter 300. Therefore, the filter 300 may be generally described as being downstream of the ozone operating system 212, the ozone outlet 230, and the exhaust fan 302. In this configuration, the exhaust fan 302 is configured urge ozone within the sanitization compartment 202 to flow through the filter 300 utilizing a blowing force generated by the exhaust fan 302.

FIG. 6 shows a schematic cross-sectional view of the sanitization device 200 of FIG. 2 taken along the line III-III, wherein the sanitization device 200 has a fourth example configuration for the filter 300 and the exhaust fan 302. As shown, according to the fourth example configuration, the ozone outlet 230 may be disposed within the ozone flow path 228 at a location between the filter 300 and the exhaust fan 302, wherein the filter 300 is within the sanitization compartment 202 and the exhaust fan 302 is external to the sanitization compartment 202. The filter 300 and the exhaust fan 302 are coupled to the sidewall 216. As such, the ozone flow path 228 extends from the ozone operating system 212 and through both the filter 300 and the ozone outlet 230 before extending through the exhaust fan 302. Therefore, the exhaust fan 302 may be generally described as being downstream of the ozone operating system 212, the ozone outlet 230, and the filter 300. In this configuration, the exhaust fan 302 is configured urge ozone within the sanitization compartment 202 to flow through the filter 300 utilizing a suction force generated by the exhaust fan 302.

FIG. 7 shows a schematic cross-sectional view of the sanitization device 200 of FIG. 2 taken along the line III-III, wherein the sanitization device 200 has a fifth example configuration for the filter 300 and the exhaust fan 302. As shown, according to the fifth example configuration, the exhaust fan 302 may be disposed within the ozone flow path 228 at a location between the ozone outlet 230 and the filter 300. The exhaust fan 302 and the filter 300 are within the sanitization compartment 202, wherein the filter 300 and the exhaust fan 302 are coupled to the sidewall 216. In other words, the filter 300 and the exhaust fan 302 are disposed within the sanitization compartment 202. As such, the ozone flow path 228 extends from the ozone operating system 212 and through both the filter 300 and the exhaust fan 302 before extending through the ozone outlet 230. Therefore, the filter 300 may be generally described as being downstream of the ozone operating system 212 and upstream of the ozone outlet 230 and the exhaust fan 302. In this configuration, the exhaust fan 302 is configured urge ozone within the sanitization compartment 202 to flow through the filter 300 utilizing a suction force generated by the exhaust fan 302.

FIG. 8 shows a schematic cross-sectional view of the sanitization device 200 of FIG. 2 taken along the line III-III, wherein the sanitization device 200 has a sixth example configuration for the filter 300 and the exhaust fan 302. As shown, according to the sixth example configuration, the filter 300 may be disposed within the ozone flow path 228 at a location between the ozone outlet 230 and the exhaust fan 302. The filter 300 and the exhaust fan 302 are within the sanitization compartment 202, wherein the filter 300 and the exhaust fan 302 are coupled to the sidewall 216. In other words, the filter 300 and the exhaust fan 302 are disposed within the sanitization compartment 202. As such, the ozone flow path 228 extends from the ozone operating system 212 and through both the exhaust fan 302 and the filter 300 before extending through the ozone outlet 230. Therefore, the exhaust fan 302 may be generally described as being downstream of the ozone operating system 212 and upstream of the ozone outlet 230 and the filter 300. In this configuration, the exhaust fan 302 is configured urge ozone within the sanitization compartment 202 to flow through the filter 300 utilizing a blowing force generated by the exhaust fan 302.

FIG. 9 shows a schematic cross-sectional view of the sanitization device 200 of FIG. 2 taken along the line III-III, wherein the sanitization device 200 has a seventh example configuration for the filter 300 and the exhaust fan 302. As shown, according the seventh example configuration, the filter 300 may be disposed within the sanitization compartment 202 at a location downstream of the exhaust fan 302 and the ozone operating system 212. For example, the exhaust fan 302 may be coupled to the connector 218 and the filter 300 may be coupled to the sidewall 216.

FIG. 10 shows a schematic cross-sectional view of the sanitization device 200 of FIG. 2 taken along the line III-III, wherein the sanitization device 200 has an eighth example configuration for the filter 300 and the exhaust fan 302. As shown, according the eighth example configuration, the exhaust fan 302 may be disposed within the sanitization compartment 202 and the filter 300 may be external to the sanitization compartment 202. For example, the exhaust fan 302 may be coupled to the connector 218 and the filter 300 may be coupled to the sidewall 216.

While FIGS. 2-10 show the ozone outlet 230 in at least one of the one or more sidewalls 216 and the connector 218 in the bottom 214, other configurations are possible. For example, the ozone outlet 230 may be in the bottom 214 and the connector 218 may be in at least one of the one or more the sidewalls 216 or the bottom 214. Further, when the exhaust fan 302 is operating, the exhaust fan 302 may encourage the circulation (or distribution) of ozone within the sanitization compartment 202. As such, in some instances, the exhaust fan 302 may operate for at least a portion of the time when the ozone operating system 212 is generating ozone. When the ozone outlet 230 is defined in the bottom 214, the filter 300 may be coupled to the bottom 214 such that, for example, ozone passing through the ozone outlet 230 passes through the filter 300.

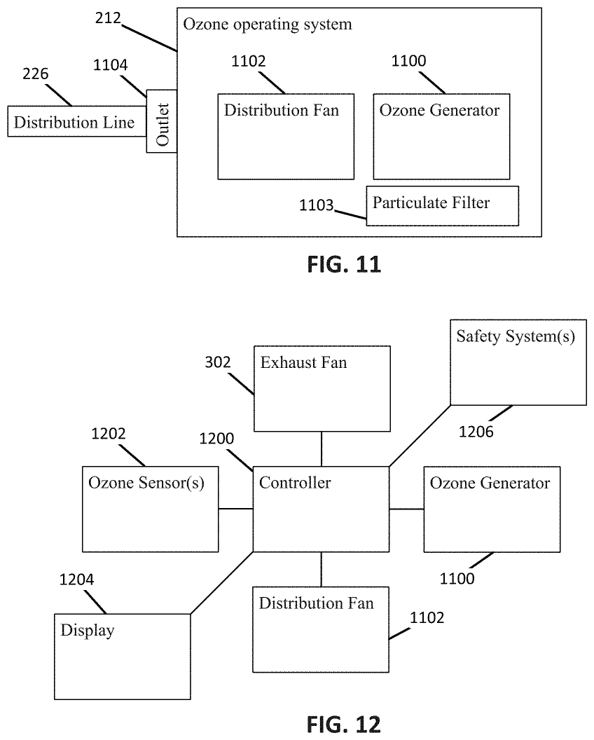

FIG. 11 shows a schematic block diagram of the ozone operating system 212, which may be an example of the ozone operating system 102 of FIG. 1. The ozone operating system 212 may include an ozone generator 1100 and a distribution fan 1102. In some instances, one or more air particulate filters 1103 may be fluidly coupled to an inlet of the ozone generator 1100, wherein the one or more air particulate filters 1103 are configured to capture at least a portion of any debris (e.g., dust) entrained within air being drawn into the ozone generator 1100. Such a configuration may mitigate or prevent ingress of debris into the ozone generator 1100 and the distribution fan 1102. In some instances, the one or more air particulate filters 1103 may be user replaceable. The ozone generator 1100 is configured to generate ozone and the distribution fan 1102 is configured to urge the generated ozone through an outlet 1104 and into the ozone distribution line 226. The distribution fan 1102 and the exhaust fan 302 can be configured to transition between operational states such that the fans 302 and 1102 cooperate during operation of the sanitization device 200.

For example, the distribution fan 1102 can be configured to transition at least between a distribution fan off state, a distribution fan distribution state, and a distribution fan exhaust state and the exhaust fan 302 can be configured to transition at least between an exhaust fan off state, an exhaust fan distribution state, and an exhaust fan exhaust state. Transitioning the distribution fan 1102 and/or the exhaust fan 302 between an off state, distribution state, and an exhaust state during use of the sanitization device 200 may improve the longevity and/or effectiveness of the filter 300 by minimizing a quantity of ozone passing therethrough while optimizing a flow rate, quantity, and/or distribution of ozone within the sanitization compartment 202. For a 12 volt (V) fan the off state may correspond to 0V, the distribution state may correspond to approximately (e.g., within 5% of) 7.2V, and the exhaust state may correspond to approximately (e.g., within 5% of) 12V.

When the ozone generator 1100 is active (generating ozone), the distribution fan 1102 can be in the distribution fan distribution state and the exhaust fan 302 can be in the exhaust fan off state, the exhaust fan distribution state, or the exhaust fan exhaust state. For example, when the ozone generator 1100 is active, the exhaust fan may be in the exhaust fan off state. Such a configuration may result in increasing a quantity of ozone within the sanitization compartment 202 (e.g., as a result of ozone not being forcibly removed from the sanitization compartment 202 through operation of the exhaust fan 302). An increased quantity of ozone within the sanitization compartment 202 may increase a rate at which an article within the sanitization compartment 202 is sanitized, assuming ozone is incident on all surfaces of the article for substantially the same time. By way of further example, when the ozone generator 1100 is active, the exhaust fan may be in the exhaust fan distribution state. Such a configuration, may encourage distribution of ozone within the sanitization compartment 202 and/or encourage ozone to be incident on obscured surfaces of an article within sanitization compartment 202. By way of still further example, when the ozone generator 1100 is active, the exhaust fan may be in the exhaust fan exhaust state. Such a configuration, may encourage distribution of ozone within the sanitization compartment 202 and/or encourage ozone to be incident on obscured surfaces of an article within sanitization compartment 202. In some instances, while the ozone generator 1100 is active, the exhaust fan 302 can be configured to transition between the exhaust fan off state and the exhaust fan distribution state and/or the exhaust fan exhaust state based on a quantity and/or distribution of ozone within the sanitization compartment 202 (e.g., as detected by one or more sensors).

When the ozone generator 1100 is inactive (discontinues generating ozone), the exhaust fan 302 can transition (e.g., from the exhaust fan distribution state) to the exhaust fan exhaust state and the distribution fan 1102 can transition (e.g., from the distribution fan distribution state) to the distribution fan off state or the distribution fan exhaust state. For example, when the ozone generator 1100 is inactive, the exhaust fan 302 can transition from the exhaust fan off state to the exhaust fan exhaust state or the exhaust fan distribution state. By way of further example, when the ozone generator 1100 is inactive, the exhaust fan 302 can transition from the exhaust fan distribution state to the exhaust fan exhaust state or remain in the exhaust fan distribution state. Configurations in which the exhaust fan 302 transitions to the exhaust fan exhaust state in response to the ozone generator 1100 becoming inactive may result in ozone within the sanitization compartment 202 being reduced to a safe quantity (e.g., less than 0.05 parts per million (ppm)) more quickly than if the exhaust fan 302 were in the exhaust fan off state or the exhaust fan distribution state.

In some instances, while the ozone generator 1100 is inactive, the distribution fan 1102 can be configured to transition between the distribution fan off state and the distribution fan exhaust state based on a quantity and/or distribution of ozone within the sanitization compartment 202 (e.g., as detected by one or more sensors). After the ozone generator 1100 is inactive for a predetermined time or the ozone within the sanitization compartment reaches a predetermined level, the exhaust fan 302 may transition (e.g., from the exhaust fan exhaust state) to the exhaust fan off state and the distribution fan 1102 may transition (e.g., from the distribution fan exhaust state) to the distribution fan off state, if not already in the distribution fan off state. In other words, the exhaust fan 302 and the distribution fan 1102 may transition to a respective off state after the ozone generator 1100 has discontinued generating ozone for a predetermined time or the ozone within the sanitization compartment 202 reaches a predetermined level. In some instances, for example, the exhaust fan 302 may transition to the exhaust fan off state after the ozone generator 1100 has discontinued generating ozone for a predetermined time or the ozone within the sanitization compartment 202 reaches a predetermined level and the distribution fan 1102 may transition to the distribution fan off state in response to the ozone generator 1100 discontinuing generation of ozone.

When transitioning from the distribution fan distribution state to the distribution fan exhaust state, a rotational speed of the distribution fan 1102 may decrease (or increase). In other words, a rotational speed of the distribution fan 1102 when in the distribution fan distribution state measures differently from a rotational speed of the distribution fan 1102 when in the distribution fan exhaust state. When transitioning from the exhaust fan distribution state to the exhaust fan exhaust state, a rotation speed of the exhaust fan 302 may increase (or decrease). In other words, a rotational speed of the exhaust fan 302 when in the exhaust fan exhaust state measures differently from a rotational speed of the exhaust fan 302 when in the exhaust fan distribution state.

By way of further example, the distribution fan 1102 may be configured to transition between at least a distribution fan distribution state and a distribution fan off state and the exhaust fan 302 may be configured to transition between at least an exhaust fan exhaust state and an exhaust fan off state. In this example, when the ozone generator 1100 is active, the distribution fan 1102 is in the distribution fan distribution state and the exhaust fan 302 is in the exhaust fan off state and, when the ozone generator 1100 is inactive, the distribution fan 1102 is in the distribution fan off state and the exhaust fan 302 is in the exhaust fan exhaust state. After the ozone generator 1100 is inactive for a predetermined time or the ozone within the sanitization compartment reaches a predetermined level, the exhaust fan 302 may transition to the exhaust fan off state.

By way of further example, the distribution fan 1102 may be configured to transition between at least a distribution fan distribution state and a distribution fan off state and the exhaust fan 302 may be configured to transition between at least an exhaust fan exhaust state and an exhaust fan off state. In this example, when the ozone generator 1100 is active and inactive, the distribution fan 1102 is in the distribution fan distribution state and the exhaust fan 302 is in the exhaust fan exhaust state for at least a portion of the time that the ozone generator 1100 is active and inactive. After the ozone generator 1100 is inactive for a predetermined time or the ozone within the sanitization compartment reaches a predetermined level, the exhaust fan 302 may transition to the exhaust fan off state and the distribution fan 1102 may transition to the distribution fan off state.

By way of further example, the distribution fan 1102 may be configured to transition between at least a distribution fan distribution state and a distribution fan off state and the exhaust fan 302 may be configured to transition between at least an exhaust fan exhaust state and an exhaust fan off state. In this example, when the ozone generator 1100 is active, the distribution fan 1102 is in the distribution fan distribution state and the exhaust fan 302 is in the exhaust fan exhaust state and, when the ozone generator 1100 is inactive, the distribution fan 1102 transitions to the distribution fan off state in response to the ozone generator 1100 becoming inactive and the exhaust fan 302 remains in the exhaust fan exhaust state for a predetermined time or until the ozone within the sanitization compartment reaches a predetermined level.

FIG. 12 shows a schematic block diagram of a controller 1200, which may control and/or monitor one or more operations of the sanitization device 200. As shown, the controller 1200 is communicatively coupled to the exhaust fan 302, the ozone generator 1100, and the distribution fan 1102. The controller 1200 can be configured to cause the ozone generator 1100 to transition between being active and inactive and to further cause the exhaust fan 302 and the distribution fan 1102 to transition between operational states.

The controller 1200 can cause the ozone generator 1100 to be active for a sanitizing time period and inactive for an exhaust time period. During the sanitizing time period, the distribution fan 1102 is caused to be in the distribution fan distribution state and the exhaust fan 302 is caused to be in one of the exhaust fan exhaust state, the exhaust fan distribution state, or the exhaust fan off state. For example, during the sanitizing time period (or when the ozone generator is generating ozone), the controller 1200 can be configured to cause the exhaust fan 302 to urge ozone through the filter 300. During the exhaust time period, the exhaust fan 302 is caused to be in the exhaust fan exhaust state and the distribution fan 1102 is caused to be in one of the distribution fan exhaust state, the distribution fan distribution state, or the distribution fan off state. For example, during the exhaust time period (or when the ozone generator 1100 has discontinued generating ozone), the controller 1200 can be configured to cause the distribution fan 1102 to urge ozone through the filter 300.

The sanitizing time period may be a predetermined time period or a dynamic time period. The predetermined time period may generally correspond to an estimated time required for the sanitization compartment 202 to have a predetermined sanitizing quantity of ozone and an estimated time required to sanitize an article within the sanitization compartment 202 using the predetermined sanitizing quantity of ozone. The dynamic time period may be based, at least in part, on a measure of a quantity of ozone within the sanitization compartment 202, as measured by one or more ozone sensors 1202. For example, the dynamic time period may generally correspond to the time required for the one or more ozone sensors 1202 to indicate that the ozone within the sanitization compartment 202 has reached a predetermined sanitizing quantity and an estimated time required to sanitize an article within the sanitization compartment 202 using the predetermined sanitizing quantity of ozone.

The predetermined sanitizing quantity of ozone may, for example, measure in a range of 125 parts per million (ppm) to 375 ppm. By way of further example, the predetermined sanitizing quantity of ozone may measure in a range of 200 ppm to 300 ppm. By way of still further example, the predetermined quantity of ozone may measure approximately (e.g., within 5% of) 250 ppm. The sanitizing time period may measure, for example, in a range of 3 minutes to 20 minutes. By way of further example, the sanitizing time period may measure approximately (e.g., within 5% of) 5 minutes. By way of still further example, the sanitizing time period may measure approximately (e.g., within 5% of) 10 minutes.

The exhaust time period may be a predetermined time period or a dynamic time period. The predetermined time period may generally correspond to an estimated time required for a quantity of ozone within the sanitization compartment 202 to be reduced to a predetermined safe quantity. The dynamic time period may be based, at least in part, on a measure of a quantity of ozone within the sanitization compartment 202, as measured by the one or more ozone sensors 1202. For example, the dynamic time period may generally correspond to the time required for the one or more ozone sensors 1202 to indicate that a quantity of ozone within the sanitization compartment 202 has been reduced to a predetermined safe quantity.

The predetermined safe quantity may measure, for example, less than 0.05 ppm. By way of further example, the predetermined safe quantity may measure 0 ppm. The exhaust time period may measure, for example, in a range of 3 minutes to 20 minutes. By way of further example, the exhaust time period may measure approximately (e.g., within 5% of) 5 minutes. By way of still further example, the exhaust time period may measure approximately (e.g., within 5% of) 10 minutes.

In some instances, the controller 1200 may be further configured to be communicatively coupled to a display 1204. The display 1204 can be a liquid crystal display (LCD), an organic light emitting diode (OLED) display, one or more light emitting diodes (LEDs) that illuminate to display a status, and/or any other type of display. The display 1204 can be configured to display a status of the sanitization device 200. Additionally, or alternatively, the controller 1200 can be configured to communicatively couple (e.g., by a wireless connection) to a personal device. For example, the controller 1200 can be configured to communicatively couple to a mobile phone disposed within the sanitization compartment 202. In this example, the display 1204 can be configured to show information relating to the mobile phone (e.g., display messages received by the mobile phone). In some instances, the display 1204 can be configured to allow a user of the sanitization device 200 to interact with the mobile phone while the mobile phone is disposed within the sanitization compartment 202. For example, a graphic user interface of the mobile phone may be rendered on the display 1204. In some instances, the display 1204 may be detachable from the sanitization device 200 and include a power supply (e.g., one or more batteries). In this instance, the display 1204 may be configured to be wirelessly communicatively coupled to the sanitization device 200. As such, when a graphic user interface of the mobile phone is rendered on the display 1204, a user may be able to interact with the mobile phone at a location remote from the sanitization device 200.

In some instances, the controller 1200 may be communicatively coupled to one or more safety systems 1206. For example, the one or more safety systems 1206 may include a lid position sensor that senses whether the lid 208 is in the closed position. In this example, when the lid position sensor indicates that the lid 208 is transitioned to the open position, the controller 1200 may prevent the ozone generator 1100 from generating ozone. In some instances, the one or more safety systems 1206 may include a lid locking mechanism configured to lock the lid 208 in the closed position during the sanitizing and exhaust time periods. Upon expiration of the exhaust time period, the lid locking mechanism may unlock the lid 208, allowing the lid 208 to transition to the open position.

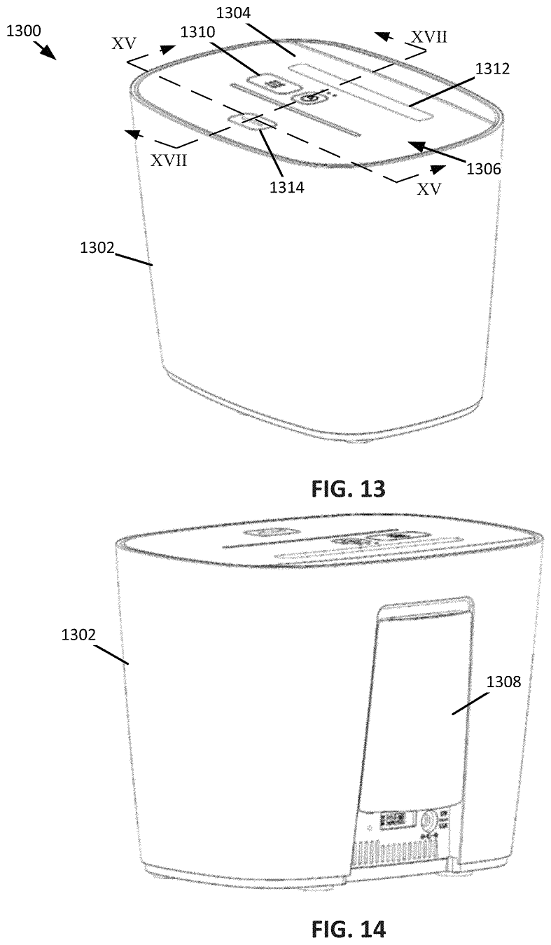

FIG. 13 shows a front perspective view of a sanitization device 1300, which may be an example of the sanitization device 200 of FIG. 2, and FIG. 14 shows a rear perspective view of the sanitization device 1300. As shown, the sanitization device 1300 includes a body 1302, an openable lid 1304 having a control panel 1306, and a filter access door 1308. The openable lid 1304 is pivotably coupled to the body 1302 such that the openable lid 1304 transitions between an open position and a closed position in response to pivotal movement. The control panel 1306 may include one or more depressible buttons 1310 configured to control one or more operations of the sanitization device 1300 (e.g., a sanitization cycle length, starting/stopping a sanitization cycle, and/or another other operation). The control panel 1306 may also include one or more displays 1312 configured to convey a status (e.g., one or more error codes, time remaining in a sanitization cycle, and/or any other status) of the sanitization device 1300 to a user. In some instances, the control panel 1306 may further include a lid release 1314. The lid release 1314 may retain the openable lid 1304 in the closed position until actuated. For example, when the openable lid 1304 is biased towards the open position (e.g., using one or more springs) the lid release 1314 may retain the openable lid 1304 in the closed position until actuated.

The filter access door 1308 is pivotally coupled to the body 1302 such that the filter access door 1308 transitions between an open position and a closed position in response to pivotal movement of the filter access door 1308. When the filter access door 1308 is in the open position, a filter of the sanitization device 1300 is accessible (e.g., for replacement). When the filter access door 1308 is in the closed position, a filter of the sanitization device 1300 is fluidly coupled to an ozone outlet of the sanitization device 1300. In some instances, ozone generation may only occur when the filter access door 1308 is in the closed position. For example, the sanitization device 1300 may include one or more sensors to determine whether the filter access door 1308 is in the open or closed position. In some instances, when ozone generation has commenced, the filter access door 1308 may be configured to be locked in the closed position to prevent the filter access door 1308 from being transitioned to the open position while ozone is being generated.

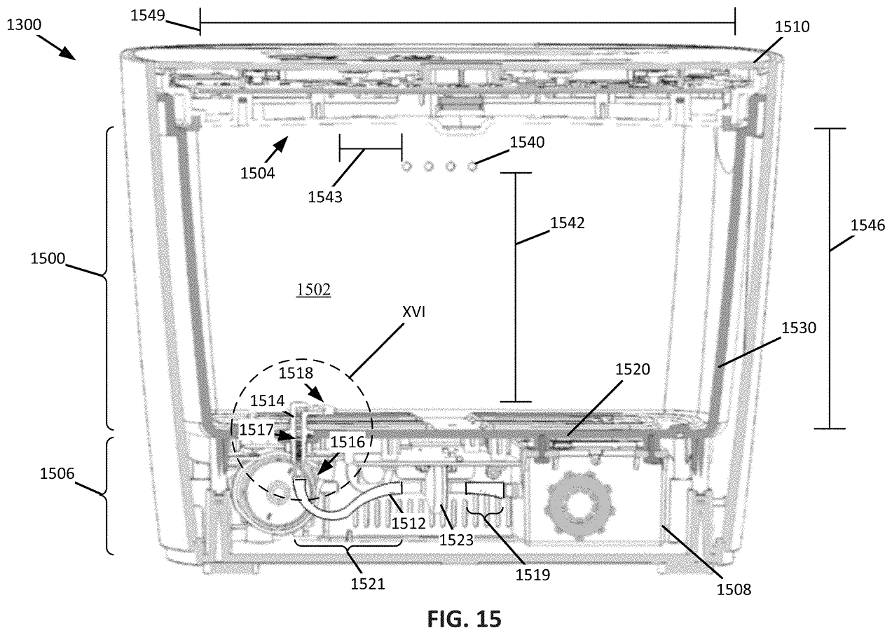

FIG. 15 shows a cross-sectional view of the sanitization device 1300 taken along the line XV-XV of FIG. 13. As shown, the sanitization device 1300 includes a sanitization compartment 1500 that defines a sanitization cavity 1502 having an open end 1504, a hardware compartment 1506 having an ozone operating system 1508, and a lid 1510 configured to selectively enclose the sanitization cavity 1502 by extending over the open end 1504. An ozone distribution line 1512 fluidly couples the sanitization compartment 1500 to the ozone operating system 1508 such that ozone generated by the ozone operating system 1508 can be urged into the sanitization compartment 1500. For example, and as shown, a connector 1514 can extend from the sanitization compartment 1500 into the hardware compartment 1506. A first end 1516 of the connector is disposed within the hardware compartment 1506 and a second end 1518 of the connector 1514 is disposed within the sanitization compartment 1500. The connector 1514 defines a fluid pathway 1517 that extends between the first and second ends 1516 and 1518. The fluid pathway 1517 is fluidly coupled to the sanitization compartment 1500 and the ozone distribution line 1512. For example, the first end 1516 of the connector 1514 can be received within the ozone distribution line 1512.

The ozone operating system 1508 (e.g., a distribution fan thereof) can be configured to urge ozone through the ozone distribution line 1512 and the fluid pathway 1517 of the connector 1514 and into the sanitization compartment 1500. As shown, the ozone distribution line 1512 may include at least a first hardware portion 1519 and a second hardware portion 1521, wherein a valve 1523 (e.g., a check valve) may be positioned between the first and second hardware portions 1519 and 1521. Additionally, or alternatively, the second end 1518 of the connector 1514 may be received within a sanitization portion of the ozone distribution line 1512 (not shown). The sanitization portion may be configured to couple to an adaptor or sanitization accessory within the sanitization compartment 1500. As such, the ozone distribution line 1512 may generally be described as being made up one or more separate portions.

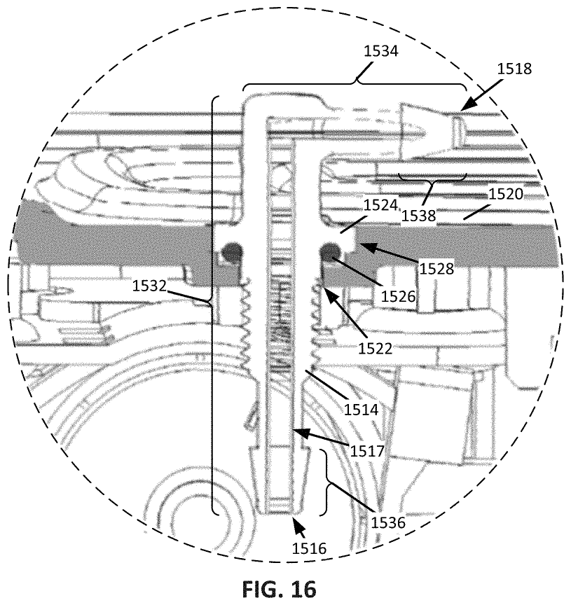

With additional reference to FIG. 16 (which shows a magnified view of the sanitization compartment 1500 generally corresponding to region XVI of FIG. 15, wherein the ozone distribution line 1512 is omitted for purposes of clarity), a base 1520 of the sanitization compartment 1500 can define a connector opening 1522 configured to receive the connector 1514. The connector 1514 may be configured to threadably engage with the connector opening 1522. In other words, the connector opening 1522 can be configured to threadably receive the connector 1514. The connector 1514 can include a flange 1524 configured to engage (directly or indirectly) with the base 1520 when the connector 1514 is completely threadably received within the connector opening 1522. For example, a seal 1526 may be disposed between the flange 1524 and the base 1520 such that, as the connector 1514 is threaded into the connector opening 1522, the seal 1526 is compressed and sealingly engages with the base 1520. In some instances, and as shown, the connector opening 1522 may be defined within a receptacle 1528. The receptacle 1528 can be configured to receive the flange 1524 and the seal 1526. As such, when the seal 1526 is compressed by the flange 1524, the seal 1526 may sealingly engage with the sides of the receptacle 1528.

The connector 1514 can be configured to direct ozone to flow along the base 1520. Ozone flowing along the base 1520 may be incident on one or more sidewalls 1530 extending from the base 1520 such that ozone is urged along and/or away from the one or more sidewalls 1530. Such a configuration may encourage ozone to flow according to a vortical motion, which may encourage distribution of the ozone within the sanitization compartment 1500. In some instances, the connector 1514 may include a first segment 1532 and a second segment 1534, wherein the second segment 1534 extends transverse to (e.g., perpendicular to) the first segment 1532. Such a configuration may result in ozone being directed toward the one or more sidewalls 1530 such that the ozone is incident on and/or flows along the one or more sidewalls 1530. As such, the connector 1514 can be generally described as being configured to encourage a distribution of ozone within the sanitization compartment 1500.

In some instances, a first barbed region 1536 may extend from the first end 1516 of the connector 1514 along at least a portion of the first segment 1532 and a second barbed region 1538 may extend from the second end 1518 along at least a portion of the second segment 1534. The first and second barbed regions 1536 and 1538 may be configured to be received within a distribution line (e.g., the ozone distribution line 1512), coupling the distribution line to the connector 1514.

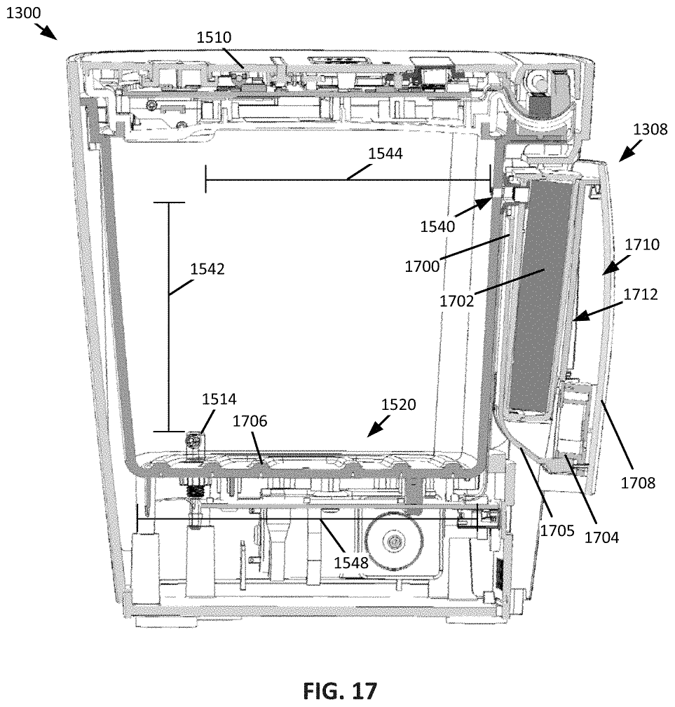

As shown, the sanitization compartment 1500 further includes at least one ozone outlet 1540. The ozone outlet 1540 is vertically spaced apart from the connector 1514 by a vertical separation distance 1542 and horizontally spaced apart from the connector 1514 by a horizontal separation distance extending lengthwise 1543 and/or widthwise 1544 (see, e.g., FIG. 17). The vertical and horizontal separation distances 1542 and 1544 may be configured to encourage distribution of ozone within the sanitization compartment 1500. As the vertical and horizontal separation distances 1542 and 1544 increase a volume of the sanitization compartment 1500 through which ozone travels before reaching the ozone outlet 1540 increases, which may encourage distribution of ozone within the sanitization compartment 1500.

A ratio of the vertical separation distance 1542 to a sanitization compartment height 1546 (i.e., the vertical separation distance 1542 divided by the sanitization compartment height 1546) may be, for example, greater than 0.25. By way of further example, the ratio of the vertical separation distance 1542 to the sanitization compartment height 1546 may be greater than 0.5. By way of still further example, the ratio of the vertical separation distance 1542 to the sanitization compartment height 1546 may be greater than 0.75. By way of still further example, the ratio of the vertical separation distance 1542 to the sanitization compartment height 1546 may be greater than 0.85. By way of still further example, the ratio of the vertical separation distance 1542 to the sanitization compartment height 1546 may be greater than 0.95. In some instances, the vertical separation distance 1542 may be maximized (e.g., the ratio of the vertical separation distance 1542 to the sanitization compartment height 1546 is greater than 0.7). The sanitization compartment height 1546 may correspond to the maximum height.

A ratio of the widthwise horizontal separation distance 1544 to a sanitization compartment width 1548 (i.e., the widthwise horizontal separation distance 1544 divided by the sanitization compartment width 1548) may be, for example, greater than 0.25. By way of further example, the ratio of the widthwise horizontal separation distance 1544 to the sanitization compartment width 1548 may be greater than 0.5. By way of still further example, the ratio of the widthwise horizontal separation distance 1544 to the sanitization compartment width 1548 may be greater than 0.75. By way of still further example, the ratio of the widthwise horizontal separation distance 1544 to the sanitization compartment width 1548 may be greater than 0.85. By way of still further example, the ratio of the widthwise horizontal separation distance 1544 to the sanitization compartment width 1548 may be greater than 0.95. In some instances, the widthwise horizontal separation distance 1544 may be maximized (e.g., the ratio of the widthwise horizontal separation distance 1544 to the sanitization compartment width 1548 is greater than 0.7). The sanitization compartment width 1548 may correspond to the maximum width.

A ratio of the lengthwise horizontal separation distance 1543 to a sanitization compartment length 1549 (i.e., the lengthwise horizontal separation distance 1543 divided by the sanitization compartment length 1549) may be, for example, greater than 0.1. By way of further example, the ratio of the lengthwise horizontal separation distance 1543 to the sanitization compartment length 1549 may be greater than 0.25. By way of still further example, the ratio of the lengthwise horizontal separation distance 1543 to the sanitization compartment length 1549 may be greater than 0.5. By way of still further example, the ratio of the lengthwise horizontal separation distance 1543 to the sanitization compartment length 1549 may be greater than 0.85. By way of still further example, the ratio of the lengthwise horizontal separation distance 1543 to the sanitization compartment length 1549 may be greater than 0.95. In some instances, the lengthwise horizontal separation distance 1543 may be maximized (e.g., the ratio of the lengthwise horizontal separation distance 1543 to the sanitization compartment length 1549 is greater than 0.7). The sanitization compartment length 1549 may correspond to the maximum width.

FIG. 17 shows a cross-sectional view of the sanitization device 1300 taken along the line XVII-XVII of FIG. 13. As shown, the filter access door 1308 includes a filter holder 1700 configured to receive a filter 1702 and further includes an exhaust fan 1704 (e.g., an axial fan having a fan inlet and a fan outlet). When the filter access door 1308 is in the closed position, the filter 1702 is fluidly coupled to the ozone outlet 1540 and, when the filter access door 1308 is in the open position, the filter 1702 is removable from the filter holder 1700 (e.g., for replacement). The filter 1702 is downstream of the connector 1514 and is configured to be fluidly coupled to the ozone outlet 1540. The exhaust fan 1704 is configured to be fluidly coupled to the ozone outlet 1540 such that the exhaust fan 1704 urges ozone to pass through the ozone outlet 1540. As shown, the exhaust fan 1704 is positioned downstream of the filter 1702 such that ozone within the sanitization compartment 1500 is drawn into the ozone outlet 1540 and through the filter 1702. At least a portion of ozone passing through the filter is reduced to oxygen that is exhausted by the exhaust fan 1704 into a surrounding environment. For example, and as shown, the filter 1702 and the exhaust fan 1704 may be downstream of the ozone outlet 1540. By way of further example, the filter 1702 may be downstream of the ozone outlet 1540 and upstream of the exhaust fan 1704. In other words, the exhaust fan 1704 is downstream of the filter 1702 and the ozone outlet 1540. The filter 1702 is configured to reduce at least a portion of ozone passing therethrough to oxygen.

The filter access door 1308 may further be configured to redirect fluid (e.g., oxygen and/or air) exiting the filter 1702 in a direction of the exhaust fan 1704. For example, and as shown, the filter access door 1308 may include (e.g., define) a fluid guide 1705. The fluid guide 1705 may have a sloped surface configured to urge a flowing fluid in a direction of a fan inlet of the exhaust fan 1704. As such, the fluid guide 1705 may generally be described as being configured to urge a fluid exhausted from the filter 1702 toward the exhaust fan 1704. The fluid may then be exhausted from a fan outlet of the exhaust fan 1704. The fan outlet may extend along a perimeter of the exhaust fan 1704 such that fluid exhausted therefrom extends in a direction transverse to (e.g., perpendicular to) a direction in which the fluid passes through an axial fan inlet.

As shown, the filter access door 1308 has a size and shape that is configured to accommodate the exhaust fan 1704 and the filter 1702 in a configuration that allows the exhaust fan 1704 to encourage a flow of ozone to pass through the filter 1702, reducing the ozone to oxygen. For example, the filter access door 1308 may include an outer wall 1708 that has an arcuate shape, wherein a concave surface of the arcuate shaped outer wall 1708 faces the filter 1702. In some instances, the outer wall 1708 can have an arcuate shape in two or more transverse (e.g., perpendicular) planes. The outer wall 1708 is spaced apart from the filter holder 1700 such that the filter 1702 and the exhaust fan 1704 are disposed within a cavity 1710 defined between the outer wall 1708 and the filter holder 1700. For example, and as shown, at least a portion of the exhaust fan 1704 can extend between the filter 1702 and the outer wall 1708 in an upright (e.g., vertical) orientation. An upright orientation of the exhaust fan 1704 may generally be described as an orientation in which an inlet side of the exhaust fan 1704 extends substantially parallel to (e.g., within 1°, 2°, 3°, 4°, or 5° of) a rear side 1712 of the filter 1702.

In some instances, when the ozone operating system 1508 (e.g., an ozone generator thereof) is generating ozone, the exhaust fan 1704 may be configured to urge ozone through the ozone outlet 1540 and the filter 1702. In other words, the exhaust fan 1704 may be active while the ozone operating system 1508 is generating ozone. By drawing ozone through the ozone outlet 1540, while ozone is being generating, the exhaust fan 1704 may encourage the distribution of ozone within the sanitization compartment 1500. In some instances, the exhaust fan 1704 may be activated after the ozone operating system 1508 has generated ozone for a predetermined time. Such a configuration may encourage distribution of ozone within the sanitization compartment 1500. When the exhaust fan 1704 is active while ozone is being generated, maximizing the vertical and horizontal separation distances 1542 and 1544 may improve the distribution of ozone within the sanitization compartment 1500.

The base 1520 can be configured such that at least a portion of an article to be sanitized is spaced apart from at least a portion of the base 1520. For example, and as shown, the base 1520 may include one or more projections 1706 that extend into the sanitization compartment 1500 in a direction of the lid 1510. The projections 1706 are configured to support an article to be sanitized such that a gap extends between at least a portion of the article be sanitized and at least a portion of the base 1520. The presence of a gap between at least a portion of the article and the base 1520 may allow ozone to pass through the gap, sanitizing the article. Additionally, or alternatively, the base 1520 may include one or more dimples. The one or more dimples may cause a gap to be formed between an article to be sanitized and the base 1520. Additionally, or alternatively, an ozone permeable material may be disposed along or spaced apart from the base 1520, wherein the ozone permeable material is configured to support an article to be sanitized. As such, when ozone passes through the ozone permeable material the surface of the article in direct contact with the ozone permeable material may have ozone incident thereon.

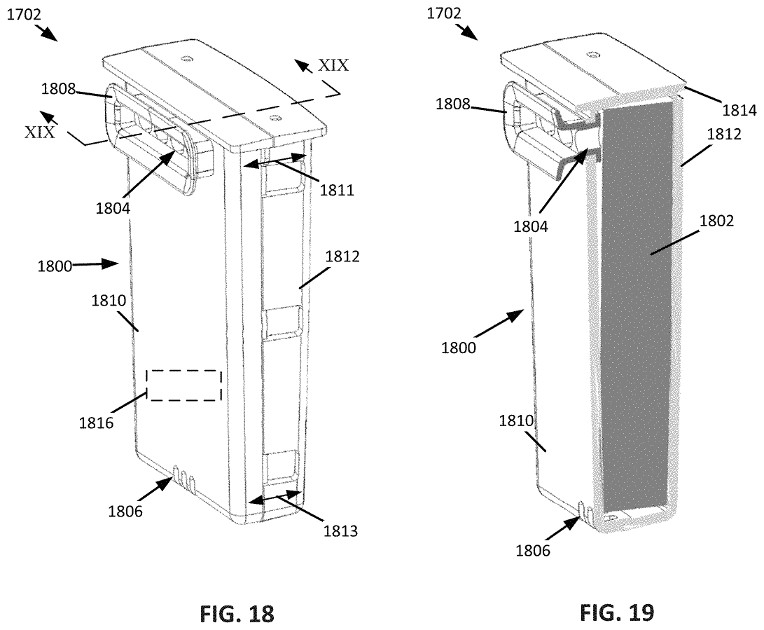

FIG. 18 shows a perspective view of the filter 1702 of FIG. 17 and FIG. 19 shows a cross-sectional perspective view of the filter 1702 taken along the line XIX-XIX of FIG. 18. As shown, the filter 1702 includes a filter housing 1800, a filter medium 1802 extending within the filter housing 1800, one or more filter inlets 1804, and one or more filter outlets 1806. An inlet seal 1808 can extend around the one or more filter inlets 1804. When the filter access door 1308 is in the closed position, the inlet seal 1808 may extend around the ozone outlet 1540 and sealingly engage with the sidewall 1530. In some instances, when in the closed position, the filter access door 1308 may be configured to urge the filter seal 1808 towards the sidewall 1530. For example, a biasing mechanism (e.g., a spring) may urge the filter access door 1308 towards the closed position. In some instances, a retaining mechanism (e.g., one or more detents) may be included to releasably retain the filter access door 1308 in the closed position. When the exhaust fan 1704 is active, ozone is caused to flow into the one or more filter inlets 1804 through the filter medium 1802. The filter medium 1802 reduces at least a portion of the ozone that is exhausted from the one or more filter outlets 1806 to oxygen.

The filter housing 1800 can be a single piece or a multipiece construction. For example, the filter housing 1800 can have a first shell 1810 configured to couple to a second shell 1812, wherein the first and second shells 1810 and 1812 define a cavity that extends therebetween for receiving the filter medium 1802. The first and second shells 1810 and 1812 can coupled together using any one or more of snap fits, adhesives, mechanical fasteners, press-fits, and/or any other form of coupling.

The filter housing 1800 may have any shape. For example, the filter housing 1800 may have a triangular shape, a rectangular shape, a pentagonal shape, a trapezoidal shape, an irregular shape, or any other shape. As shown, the filter housing 1800 may have a rectangular shape, wherein a cross-sectional width tapers from a first width 1811 proximate the one or more filter inlets 1804 to a second width 1813 proximate the one or more filter outlets 1806. As such, the first width 1811 may measure greater than the second width 1813.

The filter medium 1802 can be an ozone porous material that is configured to reduce ozone to oxygen. For example, the filter medium 1802 may be a reticulated foam that is formed from, includes, and/or is coated with a material that reduces ozone to oxygen. Non-limiting examples of such materials include activated carbon and magnesium oxide (either alone or in combination with active carbon); however, other materials may be used.

The filter medium 1802 may generally be described as having a filtration efficiency, a material density, a flow path length, a filter porosity, and a filter life. The filtration efficiency may generally be described as the ability of the filter medium 1802 to reduce ozone passing therethrough to oxygen. For example, when the filter medium 1802 has a filtration efficiency of 80%, 80% of the ozone passing through the filter medium 1802 is reduced to oxygen. By way of further example, the filter medium 1802 may have a filtration efficiency of at least 80%, at least 85%, at least 90%, at least 95%, or at least 99%. The material density of the filter medium 1802 influences the filtration efficiency. For example, increasing material density may increase the filtration efficiency (e.g., by encouraging increased interactions between ozone and the filter medium 1802). The flow path length may generally be described as the distance ozone travels through the filter medium 1802. As such, the flow path length may be a function of a length of the filter medium 1802 and a cross-sectional area of the filter medium 1802. Increasing the flow path length may increase the filtration efficiency (e.g., by encouraging increased interactions between ozone and the filter medium 1802). The filtration efficiency may further be influenced by the flow rate of ozone through the filter medium 1802. For example, decreasing the flow rate of ozone through the filter medium 1802 may increase the filtration efficiency (e.g., by encouraging increased interactions between ozone and the filter medium 1802). The filtration efficiency may also be influenced by a concentration of ozone (e.g., in a mixture of ozone and air) passing through the filter medium 1802. For example, higher concentrations of ozone may reduce the filtration efficiency. As the filter medium 1802 approaches the end of its filter life, the filtration efficiency may decrease. As such, the filter life may be based, at least in part, on the time it takes the filtration efficiency to drop below an efficiency threshold (e.g., the filtration efficiency measures less than 80%, 85%, 90%, 95%, or 99%).

In one example, the filter medium 1802 may be an activated carbon filter having a flow path length measuring 76 millimeters (mm), cross-sectional dimensions of 14 mm by 43 mm at one or more locations, and a filter life of 6-months with a daily usage of ten minutes per day, wherein the filter life is measured as the time for which the filtration efficiency is greater than 95%. In this example, the flow rate of ozone passing through the filter medium 1802 may measure in a range of 1.1 liters per minute (L/min) to 1.6 L/min and the ozone concentration may measure in a range of 150 parts-per-million (ppm) to 300 ppm. In this example, the filter porosity of the filter medium 1802 may measure in a range of 20 pores-per-inch (ppi) to 35 ppi. By way of further example, the filter porosity of the filter medium may measure 25 ppi, wherein a carbon density of the filter medium 1802 measures 1100 grams per cubic meter (g/m³). By way of still further example, the filter porosity of the filter medium may measure 30 ppi, wherein a carbon density of the filter medium 1802 measures 1600 g/m³.

The inlet seal 1808 may be formed of any material capable of forming a seal with the sidewall 1530. For example, the inlet seal 1808 may be formed of a flexible material. Such as a (e.g., low durometer) natural or synthetic polymer (e.g., silicone), rubber, and/or any other material. In some instances, the inlet seal 1808 may include a flange extending at an angle from a peripheral surface thereof, wherein the flange is configured to engage and seal with a corresponding portion of the sidewall 1530 at a location proximate to the ozone outlet 1540.

In some instances, the filter 1702 may include alignment features. The alignment features may assist a user in aligning the one or more filter inlets 1804 with the ozone outlet 1540. For example, the filter 1702 may include one or more shoulders 1814 extending from the filter housing 1800 that are configured to engage (e.g., contact) a portion of the filter access door 1308.

In some instances, the filter 1702 may include communications circuitry 1816 (shown schematically in hidden lines) configured to communicate with the sanitization device 1300. For example, the communications circuitry 1816 may be configured to transmit data verifying the integrity of the filter 1702. Such a configuration may prevent unauthorized and/or ineffective filters from being installed by a user. For example, if the integrity of the filter 1702 is unable to be validated the ozone operating system 1508 may be prevented from generating ozone and/or generation of ozone may be ceased. Validation of the integrity of the filter 1702 may include comparing the transmitted data from the filter 1702 to data stored in a memory of a controller such as controller 1200 and/or data stored remote from the controller but accessible by the controller, such as data on a remote server. In some instances, the data transmitted by the communications circuitry 1816 may be configured to confirm a presence of the filter 1702 in the filter holder 1700. If filter 1702 is determined to be in the filter holder 1700, the transmitted data may also be used to determine proper installation of the filter 1702 within the filter holder 1700. If the presence of the filter 1702 is not confirmed or the filter 1702 is determined to be improperly installed, the ozone operating system 1508 may be prevented from generating ozone. In some instances, the communications circuitry 1816 may configured to receive data from a controller (e.g., controller 1200) of the sanitization device 1300. For example, the controller of the sanitization device 1300 may be configured to transmit a filter expiration signal to the communications circuitry 1816 when the filter 1702 has been determined to have reached an end of its useful life (e.g., an efficiency with which the filter breaks down ozone has been determined to fall below a threshold). When the communications circuitry 1816 receives the filter expiration signal, the communications circuitry 1816 may be configured to transmit an expired signal such that the filter 1702 may be prevented from being used in the same or different sanitization devices 1300. For example, when the sanitization device 1300 receives the expired signal from the filter 1702, the ozone operating system 1508 may be prevented from generating ozone.

The communications circuitry 1816 may be configured to communicate wirelessly using, for example, a near field communication (NFC), BLUETOOTH®, ZIGBEE®, WIFI, or other wireless communication protocol. In some instances, the communications circuitry may include a radio-frequency identification (RFID) circuit and may be configured to communicate wirelessly using an RFID or other NFC communication protocol.

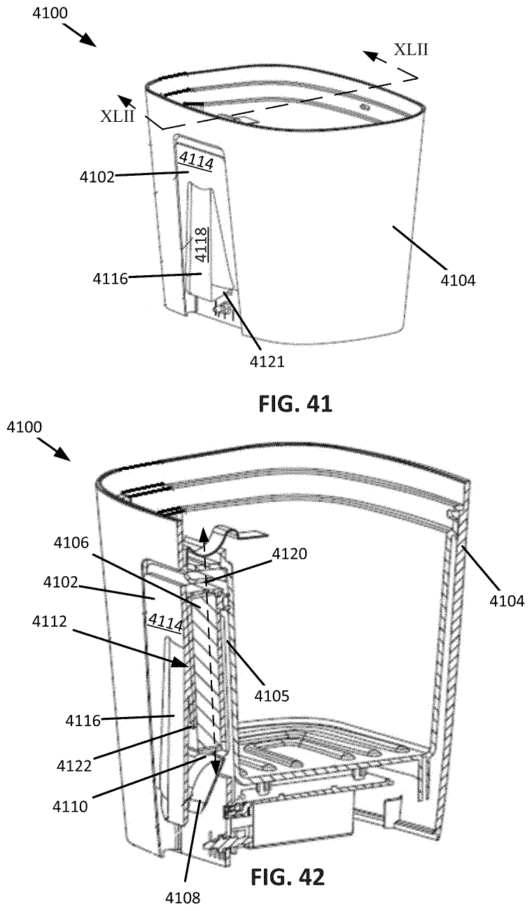

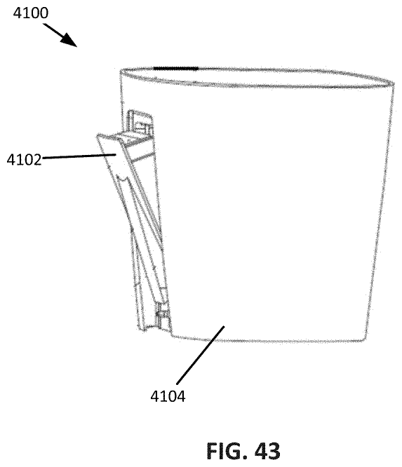

FIG. 41 shows a perspective view of a portion of a sanitization device 4100 and FIG. 42 shows a cross-sectional perspective view of the sanitization device 4100 taken along the line XLII-XLII, wherein the sanitization device 4100 may be an example of the sanitization device 200 of FIG. 2. As shown, the sanitization device 4100 includes a filter access door 4102 pivotally coupled a body 4104 of the sanitization device 4100. The filter access door 4102 is configured to transition between an open position (e.g., as shown in FIG. 43) and a closed position (e.g., as shown in FIG. 41) in response to pivotal movement of the filter access door 4102.

The filter access door 4102 includes a filter holder 4105 configured to receive a filter 4106 and further includes an exhaust fan 4108. The exhaust fan 4108 is configured to draw ozone through the filter 4106, reducing at least a portion of the ozone to oxygen, which can be exhausted into a surrounding environment. The filter access door 4102 may further include (e.g., define) a fluid guide 4110. The fluid guide 4110 can be configured to urge a fluid toward an inlet of the exhaust fan 4108.

As shown, the filter 4106 and the exhaust fan 4108 are disposed within a cavity 4112 defined between the filter holder 4102 and an outer wall 4114 of the filter access door 4102. The filter access door 4102 may further include a door extension 4116 that extends in direction away from the filter holder 4102. As shown, the door extension 4116 may include an extension wall 4118 and one or more sidewalls 4121 extending between the extension wall 4118 and the outer wall 4114 of the filter access door 4102. The door extension 4116 may generally be described as increasing a volume of the cavity 4112. Such a configuration may allow the exhaust fan 4108 to be disposed along a filter longitudinal axis 4120 of the filter 4106 (e.g., proximate an exhaust end of the filter 4106). As shown, the exhaust fan 4108 is angled relative to the filter longitudinal axis 4120, which may encourage the flow of ozone through the filter 4106. For example, the exhaust fan 4108 may be angled relative to the filter longitudinal axis 4120 by at least 2°, 3°, 4°, 5°, 10°, 15°, or more. An inlet to the exhaust fan 4108 can be an axial inlet and the outlet to the exhaust fan 4108 can be an axial outlet. Such a configuration may increase fluid flow while limiting a size of the fluid guide 4110. In some instances, a shim 4122 can be disposed between the door extension 4116 and the filter 4106.

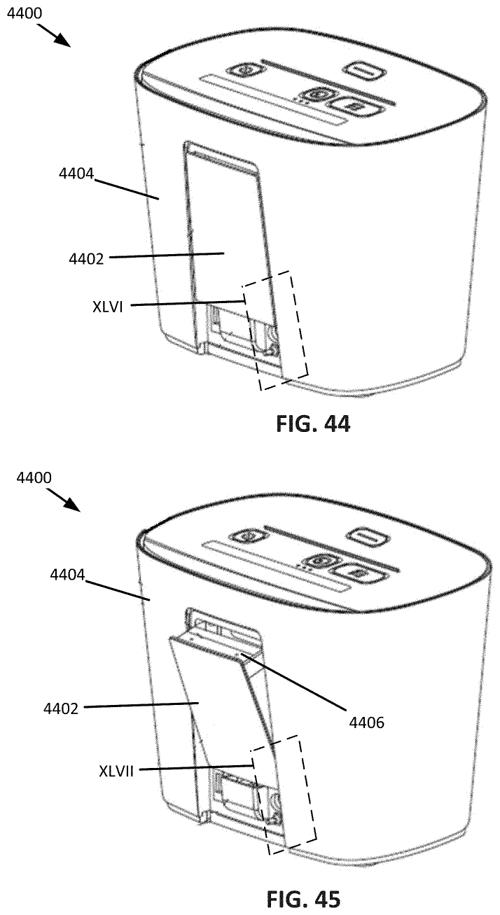

FIG. 44 shows a perspective view of a sanitization device 4400 having a filter access door 4402 in a closed position and FIG. 45 shows a perspective view of the sanitization device 4400 having the filter access door 4402 in an open position, wherein the sanitization device 4400 may be an example of the sanitization device 200 of FIG. 2. As shown, the filter access door 4402 is pivotally coupled to a body 4404 of the sanitization device 4400 such that the filter access door can transition between the open and closed positions. The filter access door 4402 is further configured to receive a filter 4406 such that the filter 4406 can be removed when the filter access door 4402 is in the open position.

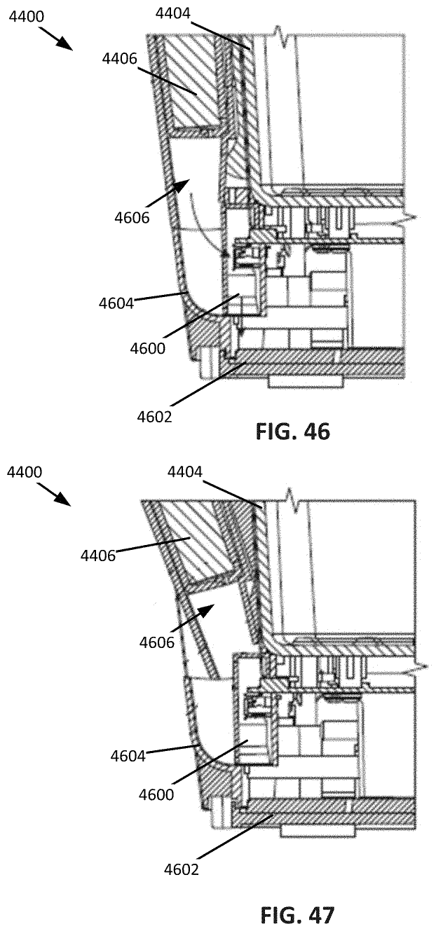

FIG. 46 is a magnified cross-sectional view generally corresponding to region XLVI of FIG. 44. As shown, an exhaust fan 4600 is positioned within a base 4602 of the body 4404 and is separate from the filter access door 4402. As such, the exhaust fan 4600 remains stationary as the filter access door 4402 transitions between the open and closed positions. Fixing the exhaust fan 4600 relative to the filter access door 4402 may improve the reliability and/or durability of power and/or control connections between the exhaust fan 4600 and a power supply and/or a controller.

The exhaust fan 4600 is configured to encourage ozone to flow through the filter 4406, which is configured to reduce ozone to oxygen, and to exhaust fluid (e.g., oxygen and/or air) into a surrounding environment. As such, the base 4602 may define one or more outlet vents that may be fluidly coupled to one or more outlet channels that are fluidly coupled to an outlet of the exhaust fan 4600. As shown, the base 4602 of the body 4404 may include (e.g., define) a fluid guide 4604. The fluid guide 4604 is configured to urge fluid (e.g., oxygen and/or air) exiting the filter 4406 towards an inlet of the exhaust fan 4600. When the filter access door 4402 is in the closed position, a cavity 4606 of the filter access door 4402 configured to receive the filter 4406 is fluidly coupled to the fluid guide 4604 such that a continuous fluid pathway extends therebetween. When the filter access door 4402 is transitioned to the open position, the fluid pathway may no longer be continuous (see, e.g., FIG. 47).

The exhaust fan 4600 can be configured to have, for example, an axial inlet and an axial outlet. By way of further example, the exhaust fan 4600 can be configured to have an axial inlet and a perimeter outlet.

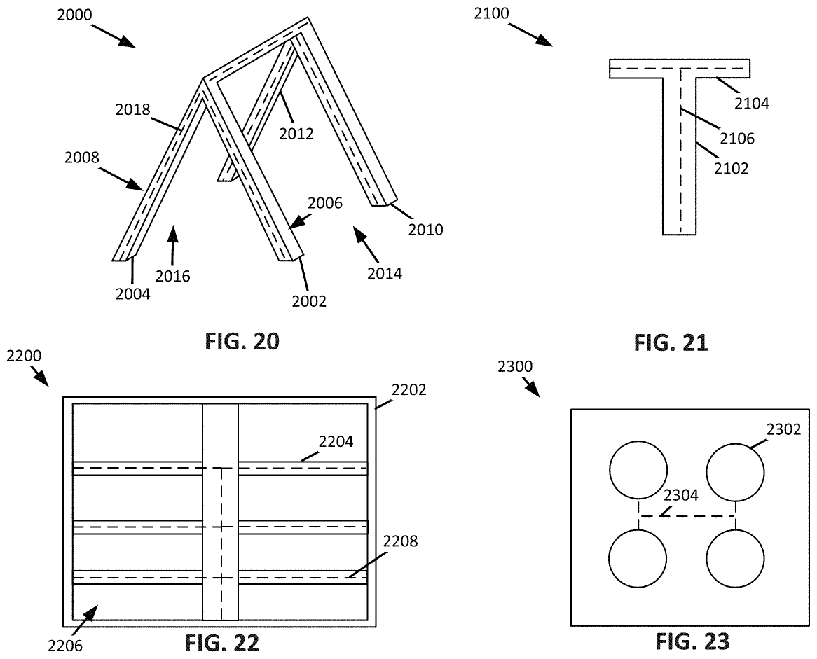

FIG. 20 shows a perspective view of a sanitization accessory 2000 configured to support one or more articles. The sanitization accessory 2000 is configured to be disposed within (e.g., removably coupled to a surface of) a sanitization compartment of a sanitization device (e.g., the sanitization compartment 104 of the sanitization device 100 of FIG. 1). As shown, the sanitization accessory 2000 may include at least a first and a second leg 2002 and 2004. The first and second legs 2002 and 2004 may extend transverse to each other. The first and second legs 2002 and 2004 may each define an article support surface 2006 and 2008, respectively. The article support surfaces 2006 and 2008 may be configured to engage the same or different articles. For example, when the article is a foldable article (e.g., a wallet, a laptop, a foldable mobile phone, and/or any other foldable article), a first portion of the foldable article may extend along the first article support surface 2006 and a second portion of the foldable article may extend along the second article support surface 2008. By way of further example, a first article may be supported by the first support surface 2006 and a second article may be supported by the second support surface 2008.

As shown, in some instances, the sanitization accessory 2000 may further include a third and a fourth leg 2010 and 2012. The third and fourth leg 2010 and 2012 may extend transverse to each other. The third leg 2010 may extend parallel to the first leg 2002 and the fourth leg 2012 may extend parallel to the second leg 2004. As shown, the third and fourth leg 2010 and 2012 may be spaced apart from the first and second legs 2002 and 2004 such that gaps 2014 and 2016 extend therebetween. The gaps 2014 and 2016 may allow ozone to pass therethrough and be incident on one or more articles supported by the sanitization accessory 2000, increasing an area of the articles exposed to ozone.

In some instances, the sanitization accessory 2000 may define one or more fluid pathways 2018 extending therein. The one or more fluid pathways 2018 may be configured to allow ozone to pass therethrough. For example, the one or more fluid pathways 2018 may be configured to be fluidly coupled to an ozone operating system (e.g., the ozone operating system 102).

FIG. 21 shows a schematic example of a sanitization accessory 2100 configured to support one or more articles. The sanitization accessory 2100 is configured to be disposed within (e.g., removably coupled to a surface of) a sanitization compartment of a sanitization device (e.g., the sanitization compartment 104 of the sanitization device 100 of FIG. 1). The sanitization accessory 2100 may include a support 2102 and one or more arms 2104 extending from the support 2102. For example, a plurality of arms 2104 may extend from the support 2102 such that the sanitization accessory 2100 has a T-shape. By way of further example, the one or more arms 2104 may extend from the support 2102 at a non-perpendicular angle. By way of still further example, the one or more arms 2104 may include at least one arcuate portion (e.g., to define a hook). The one or more arms 2104 can be configured to support an article thereon. For example, the one or more arms 2104 can be configured to support a necklace, a watch, a bracelet, and/or any other article.

In some instances, the sanitization accessory 2100 may define one or more fluid pathways 2106 extending therein. The one or more fluid pathways 2106 may be configured to allow ozone to pass therethrough. For example, the fluid pathways 2106 may be configured to be fluidly coupled to an ozone operating system (e.g., the ozone operating system 102).

FIG. 22 shows a schematic example of a sanitization accessory 2200 configured to support one or more articles. The sanitization accessory 2200 is configured to be disposed within (e.g., removably coupled to a surface of) a sanitization compartment of a sanitization device (e.g., the sanitization compartment 104 of the sanitization device 100 of FIG. 1). As shown, the sanitization accessory 2200 includes a frame 2202 having one or more shelves 2204, each defining at least a portion of a corresponding device cavity 2206. The shelves 2204 are configured to support an article such that the article is positioned within a corresponding device cavity 2206.

As shown, the frame 2202 and the one or more shelves 2204 may include one or more fluid pathways 2208 extending therein. The one or more fluid pathways 2208 are fluidly coupled to a respective device cavity 2206 and are configured to allow ozone to pass therethrough. For example, the one or more fluid pathways 2208 can be fluidly coupled to an ozone operating system (e.g., the ozone operating system 102) such that ozone can be delivered to the one or more device cavities 2206. Additionally, or alternatively, the one or more shelves 2204 may define a grate through which ozone can pass (e.g., the one or more shelves 2204 may include a plurality of openings extending therethrough) and/or include an ozone permeable material.

FIG. 23 shows a schematic example of a sanitization accessory 2300 configured to support one or more articles. The sanitization accessory 2300 is configured to be disposed within (e.g., removably coupled to a surface of) a sanitization compartment of a sanitization device (e.g., the sanitization compartment 104 of the sanitization device 100 of FIG. 1). As shown, the sanitization accessory 2300 includes one or more article openings 2302 extending therethrough. The one or more article openings 2302 are configured such that an article can be supported therein. For example, the one or more article openings 2302 can be configured to extend around a bottom portion of a corresponding article such that a substantial portion of the article extends from the corresponding article opening 2302. For example, the one or more article openings 2302 can be configured to receive a writing implement (e.g., a pen or a pencil) and support the writing implement in an upright position. In some instances, the sanitization accessory 2300 may include one or more fluid pathways 2304, each fluidly coupled to a corresponding article opening 2302. The fluid pathways 2304 are fluidly coupled to an ozone operating system (e.g., the ozone operating system 102) such that ozone can flow from the fluid pathways 2304 and through the one or more article openings 2302.

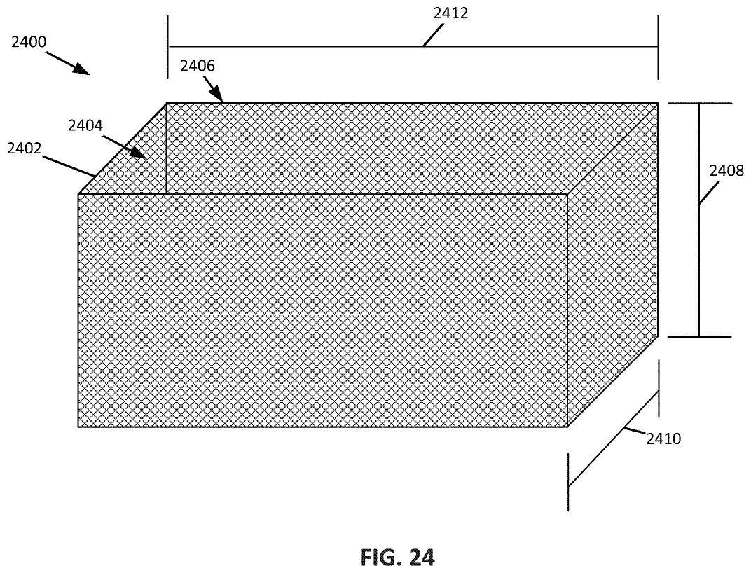

FIG. 24 shows a schematic example of a sanitization accessory 2400 configured to support one or more articles. The sanitization accessory 2400 is configured to be disposed within (e.g., removably coupled to a surface of) a sanitization compartment of a sanitization device (e.g., the sanitization compartment 104 of the sanitization device 100 of FIG. 1). As shown, the sanitization accessory 2400 can include one or more mesh walls 2402 that define an accessory cavity 2404 having at least one open end 2406 configured to receive an article. When the article is received within the accessory cavity 2404, the article may rest on one or more of the mesh walls 2402 (e.g., a mesh wall 2402 defining a base of the accessory cavity 2404). The one or more mesh walls 2402 may include a plurality of rods/bars that are spaced apart from one another. The one or more mesh walls 2402 may be made of metal (e.g., a stainless steel alloy, an aluminum alloy, and/or any other metal or metal alloy), plastic, glass, and/or any other material. For example, the one or more mesh walls 2402 may include a first plurality of metal rods/bars that extend parallel to each other and that are spaced apart from each other by a separation distance. In this example, the one or more mesh walls 2402 may further include a second plurality of metal rods/bars that are spaced apart from each other and that extend transverse to (e.g., perpendicular to) the first plurality of metal rods/bars. By way of further example, the one or more mesh walls 2402 may be at least partially defined by 3 millimeter (mm) rods of a stainless steel alloy.

The sanitization accessory 2400 may have an accessory height 2408, an accessory width 2410, and an accessory length 2412. The accessory height, width, and length 2408, 2410, and 2412 may measure less than or equal to a corresponding dimension of a sanitization compartment of a sanitization device. For example, a ratio of the accessory height 2408 to a corresponding sanitization compartment height (i.e., the accessory height 2408 divided by the sanitization compartment height) may be approximately (e.g., within 5% of) ⅓. By way of further example, a ratio of the accessory width 2410 to a corresponding sanitization compartment width (i.e., the accessory width 2410 divided by the sanitization compartment width) may be approximately (e.g., within 5% of) 1. By way of still further example, a ratio of the accessory length 2412 to a corresponding sanitization compartment length (i.e., the accessory length 2412 divided by the sanitization compartment length) may be approximately (e.g., within 5% of) 1.

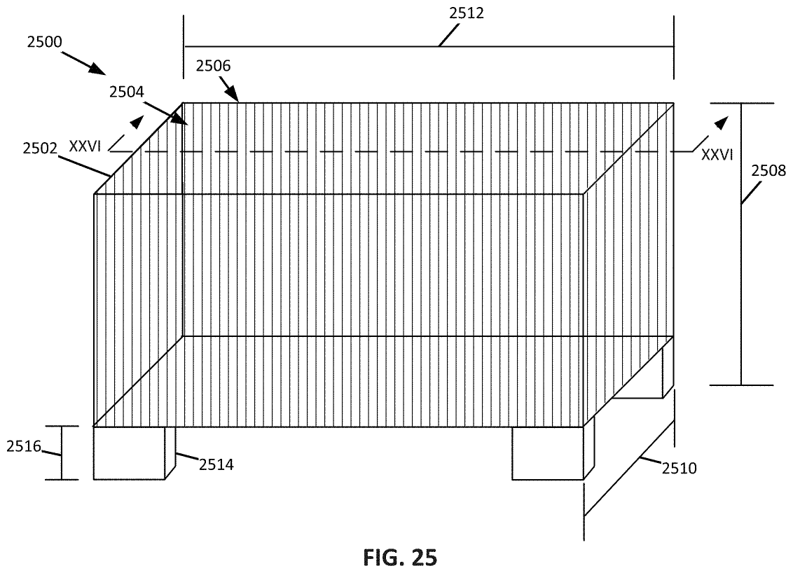

The sanitization accessory 2400 may be configured such that the sanitization accessory 2400 does not interfere with a connector (e.g., the connector 218) configured to deliver ozone to the sanitization compartment. For example, one or more standoffs may extend between a corresponding mesh wall 2402 and a base of the sanitization compartment, wherein the standoffs are configured to space the corresponding mesh wall 2402 apart from the connector. The standoffs may be coupled to or formed from the sanitization accessory 2400 or the sanitization compartment. By way of further example, at least one mesh wall 2402 (e.g., a mesh wall 2402 that defines a base of the sanitization accessory 2400) may include an opening configured to allow the connector to pass therethrough. By way of still further example, the sanitization accessory 2400 may be disposed within an additional sanitization accessory such as, for example, the sanitization accessory 2500 of FIG. 25. The additional sanitization accessory may be configured to space the sanitization accessory 2400 apart from the connector such that the sanitization accessory 2400 does not interfere with the connector.

FIG. 25 shows a schematic example of the sanitization accessory 2500 configured to support one or more articles. As discussed in relation to FIG. 24, the sanitization accessory 2500 may be configured to receive one or more additional sanitization accessories (e.g., the sanitization accessory 2400). As such, when the sanitization accessory 2500 receives the sanitization accessory 2400, the sanitization accessory 2500 may generally be described as being a first sanitization accessory and the sanitization accessory 2400 may generally be described as being a second sanitization accessory. Such a configuration allows articles within the first sanitization accessory 2500 to be separated from articles within the second sanitization accessory 2400. For example, in some instances, the first sanitization accessory 2500 may generally be referred to as a first sanitization basket and the second sanitization accessory 2400 may be generally referred to as a second sanitization basket.

As shown, the sanitization accessory 2500 can include one or more mesh walls 2502 that define an accessory cavity 2504 having at least one open end 2506 configured to receive an article. When the article is received within the accessory cavity 2504, the article may rest on one or more of the mesh walls 2502 (e.g., a mesh wall 2502 defining a base of the accessory cavity 2504). The one or more mesh walls 2502 may include a plurality of rods/bars that are spaced apart from one another. The one or more mesh walls 2502 may be made of metal (e.g., a stainless steel alloy, an aluminum alloy, and/or any other metal or metal alloy), plastic, glass, and/or any other material. For example, the one or more mesh walls 2502 may include a first plurality of metal rods/bars that extend parallel to each other and that are spaced apart from each other by a separation distance. In this example, the one or more mesh walls 2502 may further include a second plurality of metal rods/bars that are spaced apart from each other and that extend transverse to (e.g., perpendicular to) the first plurality of metal rods/bars. By way of further example, the one or more mesh walls 2502 may be at least partially defined by 3 mm rods of a stainless steel alloy.

The sanitization accessory 2500 may have an accessory height 2508, an accessory width 2510, and an accessory length 2512. The accessory height, width, and length 2508, 2510, and 2512 may measure less than or equal to a corresponding dimension of a sanitization compartment of a sanitization device. For example, a ratio of the accessory height 2408 to a corresponding sanitization compartment height (i.e., the accessory height 2508 divided by the sanitization compartment height) may be approximately (e.g., within 5% of) ⅔. By way of further example, a ratio of the accessory width 2510 to a corresponding sanitization compartment width (i.e., the accessory width 2510 divided by the sanitization compartment width) may be approximately (e.g., within 5% of) 1. By way of still further example, a ratio of the accessory length 2512 to a corresponding sanitization compartment length (i.e., the accessory length 2512 divided by the sanitization compartment length) may be approximately (e.g., within 5% of) 1.

The sanitization accessory 2500 may be configured such that the sanitization accessory 2500 does not interfere with a connector (e.g., the connector 218) configured to deliver ozone to the sanitization compartment. For example, one or more standoffs 2514 may extend between a corresponding mesh wall 2502 and a base of the sanitization compartment, wherein the standoffs 2514 are configured to space the corresponding mesh wall 2502 apart from the connector. The standoffs 2514 may have a standoff height 2516 that measures equal to or greater than a height of the connector. For example, the standoff height 2516 may measure at least 15 mm, 10 mm, 5 mm, or 1 mm greater than the height of the connector. By way of further example, the standoff height 2516 may measure approximately (e.g., within 5% of) 5 mm greater than the height of the connector. The standoffs 2514 may be coupled to or formed from the sanitization accessory 2500 or the sanitization compartment. By way of further example, at least one mesh wall 2502 (e.g., a mesh wall 2502 that defines a base of the sanitization accessory 2500) may include an opening configured to allow the connector to pass therethrough.

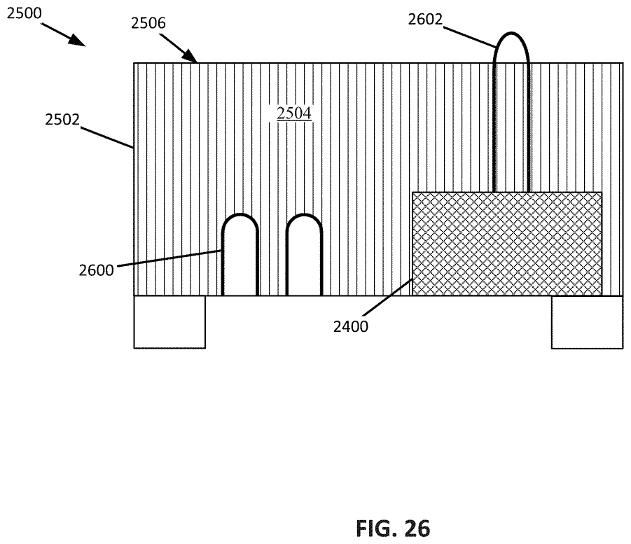

FIG. 26 shows a cross-sectional view of the first sanitization accessory 2500 taken along the line XXVI-XXVI, wherein the second sanitization accessory 2400 is disposed within the first accessory cavity 2504. As shown, the first sanitization accessory 2500 includes one or more supports 2600 that extend from a corresponding mesh wall 2502 (e.g., the mesh wall 2502 defining the base of the sanitization accessory 2400) and into the first accessory cavity 2504. The one or more supports 2600 are configured to support an article within the first accessory cavity 2504. For example, an article such, as a personal electronic device (e.g., a mobile phone), may be positioned between a plurality of supports 2600 such that the supports 2600 cooperate to support the article in an upright position. Such a configuration may increase a quantity of articles that can be positioned within the accessory cavity 2504 without significantly obscuring one or more surfaces of the articles (e.g., at least 80% of the surface area of each external surface of the article is unobscured).

As shown, in some instances, the second sanitization accessory 2400 may include a handle 2602. The handle 2602 can be configured to extend from the open end 2506 of the first accessory cavity 2504 and remain within the sanitization compartment. The handle 2602 may allow the second sanitization accessory 2400 to be more easily positioned within and/or removed from the first accessory cavity 2504. As also shown, the second mesh wall 2402 may have a different configuration from the first mesh wall 2502. For example, the second mesh wall 2402 may include a first plurality of rods/bars that extend parallel to each other and a second plurality of rods/bars that extend parallel to each other and transverse to the first plurality of rods/bars and the first mesh wall 2502 may include only a first plurality of bars/rods that extend parallel to each other.

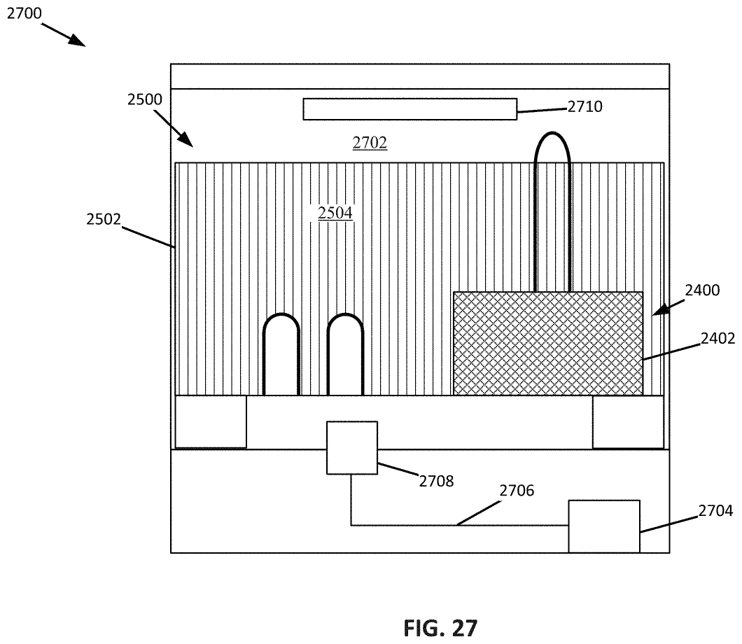

FIG. 27 shows a schematic cross-sectional view of a sanitization device 2700, which may be an example of the sanitization device 200 of FIG. 2. As shown, the first sanitization accessory 2500 is positioned within a sanitization compartment 2702 of the sanitization device 2700 and the second sanitization accessory 2400 is positioned within the first accessory cavity 2504 of the first sanitization accessory 2500. The sanitization device 2700 includes an ozone operating system 2704 configured to urge ozone through an ozone distribution line 2706 and a connector 2708 and into the sanitization compartment 2702. Once the ozone enters the sanitization compartment 2702, at least a portion of the ozone passes through the mesh walls 2502 and 2402 of the first and second sanitization accessories 2500 and 2400 before passing through an ozone outlet 2710. As shown, the first mesh walls 2502 of the first sanitization accessory 2500 are vertically spaced apart from the connector 2708. As such, at least a portion of the connector 2708 can extend into the sanitization compartment 2702.

While FIG. 27 shows the sanitization device 2700 having the sanitization accessories 2400 and 2500 disposed within the sanitization compartment 2702, any sanitization accessory may be disposed within the sanitization compartment 2702. For example, any one or more of the sanitization accessories 2000, 2100, 2200, and/or 2300 may be disposed within the sanitization compartment 2702 of the sanitization device 2700.

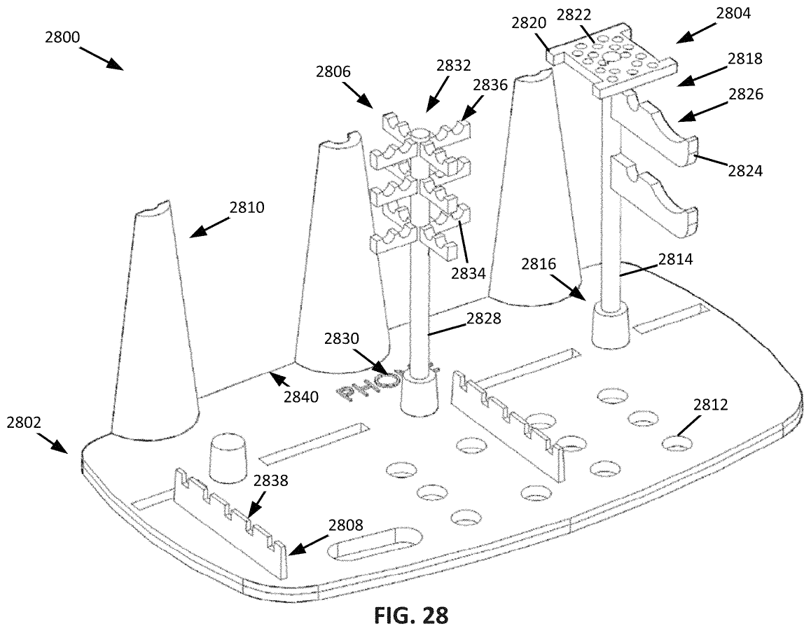

FIG. 28 shows a perspective view of a modular sanitization accessory 2800. The modular sanitization accessory 2800 may be made of any material including, for example, one or more of a metal alloy (e.g., an aluminum alloy or stainless steel alloy), plastic, and/or any other material. For example, the modular sanitization accessory 2800 may be at least partially formed through injection molded plastic. The modular sanitization accessory 2800 may be configured to support one or more articles to be sanitized (e.g., one or more of personal electronic devices, eyewear, writing implements, jewelry, keychains, personal time pieces, paper/coin money, credit cards, and/or any other article).

As shown, the modular sanitization accessory 2800 includes a platform 2802, a first accessory rack 2804 extending from the platform 2802, a second accessory rack 2806 extending from the platform 2802, one or more accessory risers 2808 extending from the platform 2802, and one or more accessory spacers 2810 extending from the platform 2802. One or more of the first accessory rack 2804, the second accessory rack 2806, the one or more accessory risers 2808, and/or the one or more accessory spacers 2810 may be removably coupled to the platform 2802 such that a position, relative to the platform 2802, of one or more of the first accessory rack 2804, the second accessory rack 2806, the one or more accessory risers 2808, and/or the one or more accessory spacers 2810 may be adjusted. As such, the first accessory rack 2804, the second accessory rack 2806, the one or more accessory risers 2808, and/or the one or more accessory spacers 2810 may, in some instances, be collectively referred to as modular accessory components.

The platform 2802 may further include a plurality of platform openings 2812. The platform openings 2812 are configured to removably receive a portion of a corresponding one of the first accessory rack 2804, the second accessory rack 2806, and/or the one or more accessory risers 2808. For example, the platform openings 2812 may be configured to form a friction fit with a portion of a corresponding one of the first accessory rack 2804, the second accessory rack 2806, and/or the one or more accessory risers 2808. By way of further example, the platform openings 2812 may be configured to threadably couple to a portion of a corresponding one of the first accessory rack 2804, the second accessory rack 2806, and/or the one or more accessory risers 2808. As shown, the platform openings 2812 may have a circular and/or rectangular cross-section. However, the platform openings 2812 may have any cross-section.

The first accessory rack 2804 includes a first rack support 2814 having a first rack mounting end region 2816 and a first rack distal end region 2818 that is opposite the first rack mounting end region 2816. At least a portion of the first rack mounting end region 2816 is configured to be received within a corresponding platform opening 2812. The first rack distal end region 2818 may include an article plate 2820 configured to support an article to be sanitized thereon (e.g., a watch, a bracelet, or a necklace). The article plate 2820 may include one or more plate openings 2822 through which ozone can pass. The first accessory rack 2804 may further include one or more first rack arms 2824 extending from the first rack support 2814. The one or more first rack arms 2824 may include one or more first arm recesses 2826 configured to receive one or more articles to be sanitized (e.g., jewelry, such as a necklace, a ring, or a bracelet, or eyewear, such as sunglasses, eye-glasses, or safety glasses). The one or more first arm recesses 2826 may have an arcuate, triangular, square, or any other shape. In some instances, the first arm recesses 2826 may be configured to support specific articles to be sanitized (e.g., jewelry or eyewear).

The second accessory rack 2806 includes a second rack support 2828 having a second rack mounting end region 2830 and a second rack distal end region 2832 that is opposite the second rack mounting end region 2830. At least a portion of the second rack mounting end region 2830 is configured to be received within a corresponding platform opening 2812. The second accessory rack 2806 may include one or more second rack arms 2834 extending from the second rack support 2828. The one or more second rack arms 2834 may include one or more second arm recesses 2836 configured to receive one or more articles to be sanitized (e.g., jewelry such as a necklace, a ring, or a bracelet). The one or more second arm recesses 2836 may have an arcuate, triangular, square, or any other shape.

The one or more accessory risers 2808 may include one or more riser recesses 2838 configured to receive a portion of an article to be sanitized (e.g., a writing implement, such as a pen or pencil, eyewear such as eye-glasses, sunglasses, or safety glasses, and/or any other article to be sanitized capable of being supported thereby). For example, and as shown, the platform 2802 may include a plurality of accessory risers 2808. The plurality of accessory risers 2808 may be spaced apart from and extend parallel to each other, wherein at least one of the one or more riser recesses 2838 of the first accessory riser 2808 is aligned with a corresponding riser recess 2838 of the second accessory riser 2808. As such, an article to be sanitized (e.g., a writing implement, such as a pen or pencil) may be supported within the aligned riser recesses 2838 of the accessory risers 2808. The one or more riser recesses 2838 may have an arcuate, triangular, square, or any other shape.

The one or more accessory spacers 2810 extend from the platform 2802. For example, at least a portion of the one or more accessory spacers 2810 may extend along a perimeter 2840 of the platform 2802. The accessory spacers 2810 are configured to space an article to be sanitized (e.g., a mobile phone, a tablet computer, a card such as credit card, or a book) apart from the perimeter 2840. As such, when the modular sanitization accessory 2800 is received within a sanitization compartment of a sanitization device (e.g., the sanitization compartment 104 of the sanitization device 100 of FIG. 1), the article to be sanitized is spaced part from a sidewall of the sanitization compartment. The one or more accessory spacers 2810 may have a conical or frustoconical shape that tapers from a widest point proximate the platform 2802 to a narrowest point that is spaced apart from the platform 2802. In other words, the one or more spacers may have a conical or frustoconical shape that tapers in a direction away from the platform 2802. When the accessory spacers 2810 have a conical or frustoconical shape that tapers in a direction away from the platform 2802, an article supported thereby may be supported at angle. The curvature of the conical or frustoconical accessory spacers 2810 may minimize a size of a contact point between a corresponding accessory spacer 2810 and an article supported thereby.

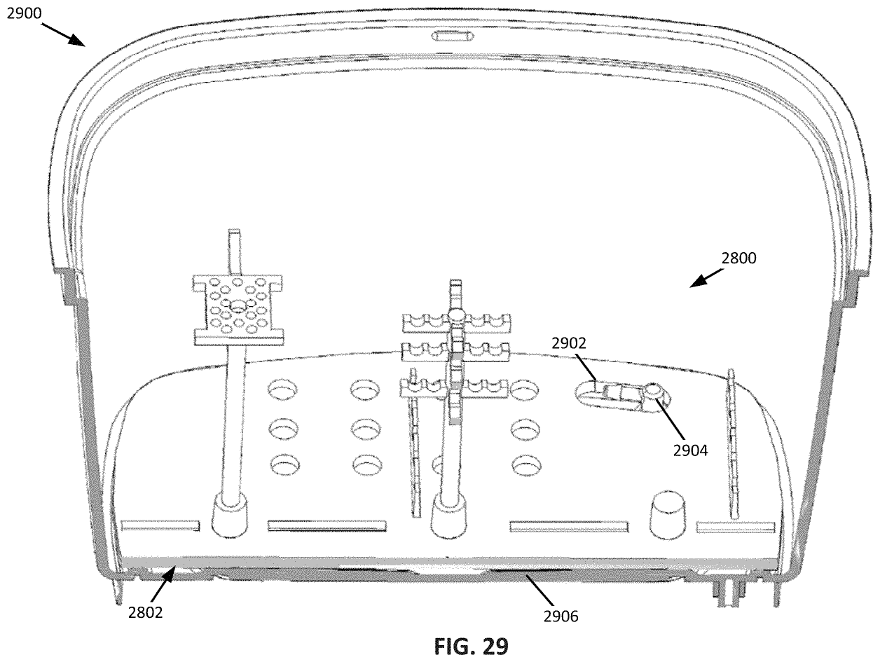

FIG. 29 shows a cross-sectional perspective view of a portion of a sanitization compartment 2900, which may be an example of the sanitization compartment 104 of FIG. 1, having the modular sanitization accessory 2800 disposed therein. As shown, a shape of the platform 2802 generally corresponds to a shape of the sanitization compartment 2900. As also shown, the platform 2802 includes a connector opening 2902 through which a connector 2904 extends. As such, in operation, ozone passing through the connector 2904 is urged from the connector 2904 in a direction along the platform 2802. As also shown, the platform 2802 may be supported by a base 2906 of the sanitization compartment 2900.

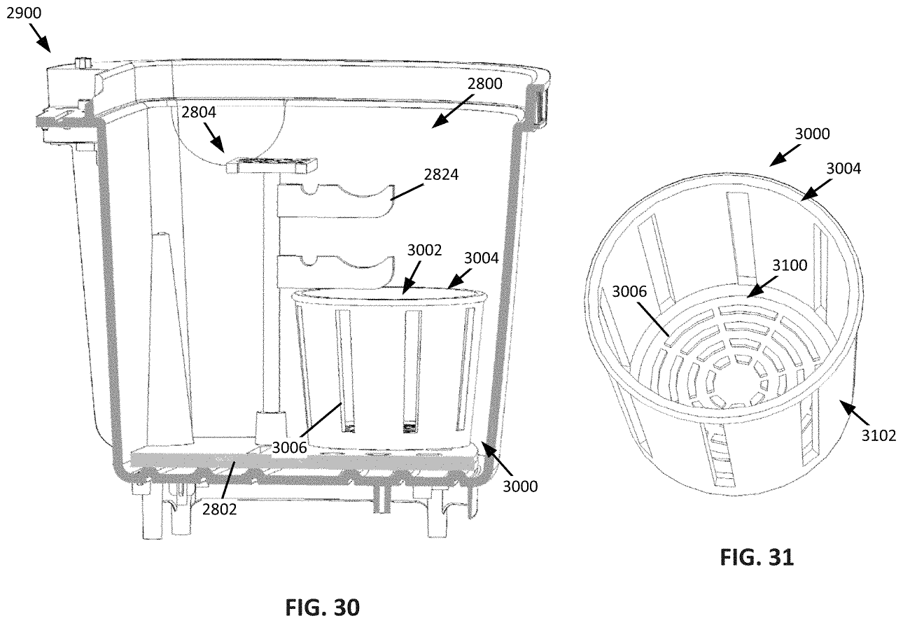

FIG. 30 shows a perspective cross-sectional view of a portion of the sanitization compartment 2900 having the modular sanitization accessory 2800 and a basket 3000 disposed therein. The basket 3000 is configured to receive one or more articles to be sanitized (e.g., keys, coins, cards such as credit cards, or eyewear) into a basket cavity 3002 having at least one basket open end 3004. As shown, the basket 3000 is supported on the platform 2802 and includes one or more basket openings 3006 through which ozone can pass. The basket 3000 can be configured such that the basket 3000 can be positioned between the one or more first rack arms 2824 of the first accessory rack 2804 and the platform 2802. In some instances, the basket 3000 can be configured to removably couple to the platform 2802 (e.g., using a plurality of magnets). FIG. 31 shows a perspective top view of the basket 3000. As shown, the basket 3000 may include a plurality of basket openings 3006 wherein at least one basket opening 3006 is defined in a first sidewall 3100 defining a basket base and at least one basket opening 3006 is defined in a second sidewall 3102 extending from the first sidewall 3100. As also shown, the first sidewall 3100 is opposite the basket open end 3004. The basket 3000 may be made of any material including, for example, one or more of a metal alloy (e.g., an aluminum alloy or stainless steel alloy), plastic, and/or any other material.

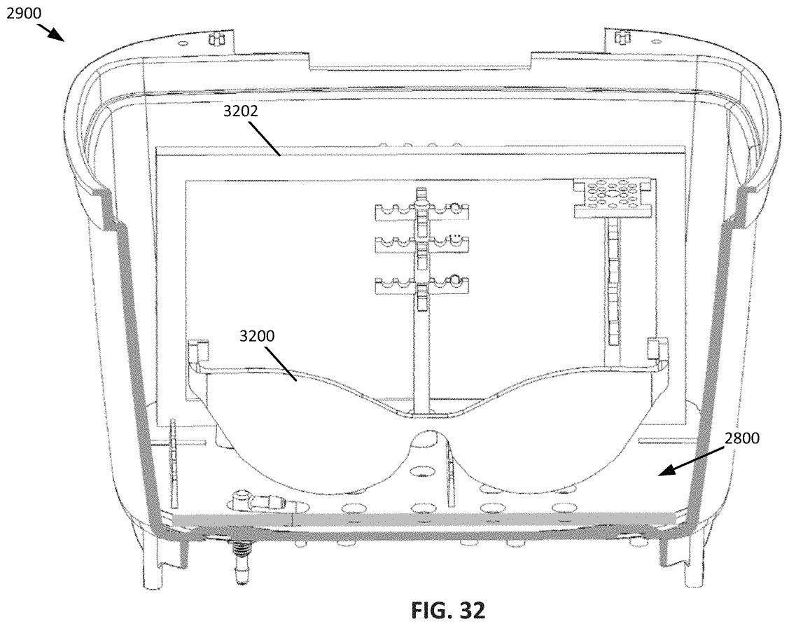

FIG. 32 shows a perspective cross-sectional view of a portion of the sanitization compartment 2900 having the modular sanitization accessory 2800, a first article to be sanitized 3200, and a second article to be sanitized 3202 disposed therein. As shown, the first article to be sanitized 3200 may be eyewear (e.g., sunglasses, eyeglasses, or safety glasses) and the second article to be sanitized 3202 may be an electronic device (e.g., a mobile phone or tablet computer).

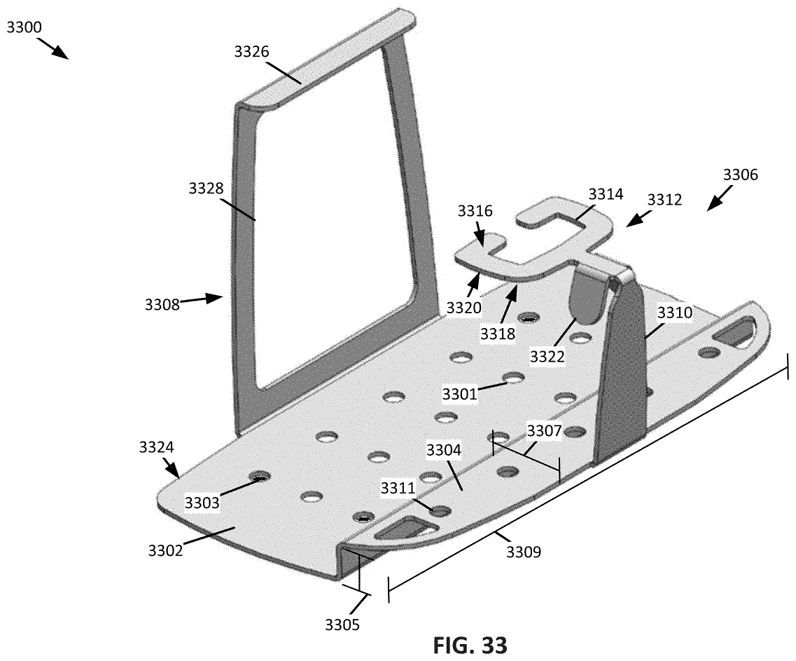

FIG. 33 shows a perspective view of a sanitization accessory 3300. The sanitization accessory 3300 includes a platform 3302, a step 3304, an accessory rack 3306, and an accessory spacer 3308. The sanitization accessory 3300 may be a monolithic body. In other words, the sanitization accessory 3300 may be formed of a single piece of material (e.g., a metal alloy, such as a stainless steel alloy or an aluminum alloy, or a plastic). For example, the sanitization accessory 3300 may be formed from a single sheet of a metal alloy, wherein the single sheet of metal alloy is cut according to a pattern such that the single sheet of metal alloy can be bent into a three dimensional shape that defines the features of sanitization accessory 3300. By way of further example, the sanitization accessory 3300 may be formed by injection molding.

The platform 3302 may include one or more platform openings 3301 extending therethrough. The one or more platform openings 3301 may be configured to allow ozone to pass therethrough. In some instances, at least one of the one or more platform openings 3301 may be configured to receive a standoff 3303 (shown schematically). The standoff 3303 may be configured to space the platform 3302 apart from a base of a sanitization compartment of a sanitization device (e.g., the sanitization compartment 104 of the sanitization device 100 of FIG. 1). The standoff 3303 may be made of a plastic, a rubber, and/or any other material. The standoff 3303 may be configured to resist a sliding movement of the platform 3302 relative to the sanitization compartment. For example, the standoff 3303 may include rubber, wherein the rubber is configured to directly engage with a portion of the sanitization compartment. Additionally, or alternatively, the standoff 3303 may extend from a corresponding platform opening 3301 and in a direction away from the base of the sanitization compartment such that an article to be sanitized engages the standoff 3303. For example, the standoff 3303 may be configured to engage the article to be sanitized such that sliding movement of the article to be sanitized, relative to the platform 3302, is resisted as a result of the engagement. In some instances, for example, an article to be sanitized that is supported by the accessory spacer 3308 may be supported in an at least partially upright position as a result of an engagement of the article to be sanitized with the accessory spacer 3308 and at least one standoff 3303. In some instances, and by way of further example, an article to be sanitized may be at least partially supported by one or more standoffs 3303 such that at least a portion of the article to be sanitized is spaced apart from the platform 3302.

The step 3304 is configured such that, when received within a sanitization compartment of a sanitization device (e.g., the sanitization compartment 104 of the sanitization device 100 of FIG. 1), the sanitization accessory 3300 does not interfere with a connector (e.g. the connector 218 of FIG. 2) configured to distribute ozone within the sanitization compartment. As such, a step height 3305 may measure equal to or greater than a corresponding connector height, a step width 3307 may measure equal to or greater than a corresponding connector width, and a step length 3309 may measure equal to or greater than a corresponding connector length. The step 3304 may include one or more step openings 3311 configured to allow ozone to pass therethrough.

The accessory rack 3306 includes a rack support 3310 extending from the step 3304 and an article plate 3312 extending from the rack support 3310. The article plate 3312 may include one or more plate openings 3314 defined within the article plate 3312. The one or more plate openings 3314 may extend from a supporting surface 3316 and through a platform facing surface 3318 of the article plate 3312. At least one of the one or more plate openings 3314 may extend from the supporting surface 3316 through the platform facing surface 3318 and through a lateral surface 3320, wherein the lateral surface 3320 extends between the supporting surface 3316 and the platform facing surface 3318. When at least one of the one or more plate openings 3314 extends through the lateral surface 3320, a portion of an article to be sanitized (e.g., a ring, a keyring, a bracelet, watch, or a necklace) can be received within the plate opening 3314 and supported by the article plate 3312 in a suspended orientation.

The article plate 3312 may further include one or more plate arms 3322 that extend away from the article plate 3312 and in a direction of the platform 3302. In some instances, the article plate 3312 may be configured to support eyewear at a position spaced apart from the platform 3302. For example, the article plate 3312 may include a plurality of plate arms 3322, wherein the plate arms 3322 are configured to support one or more nose pieces corresponding to eyewear positioned on the article plate 3312. In this example, the temple portion (or ear pieces) of the eyewear may extend over the one or more plate openings 3314.

The accessory spacer 3308 extends from the platform 3302 and may extend along a perimeter 3324 of the platform 3302. The accessory spacer 3308 is configured to space an article to be sanitized (e.g., a mobile phone, a tablet computer, a card such as credit card, or a book) apart from the perimeter 3324. As such, when the sanitization accessory 3300 is received within a sanitization compartment of a sanitization device (e.g., the sanitization compartment 104 of the sanitization device 100 of FIG. 1), the article to be sanitized is spaced part from a sidewall of the sanitization compartment. The accessory spacer 3308 may further include a handle 3326 and one or more spacer openings 3328. The handle 3326 may facilitate removal of the sanitization accessory 3300 from a sanitization compartment of a sanitization device. The one or more spacer openings 3328 may be configured such that ozone can pass therethrough.

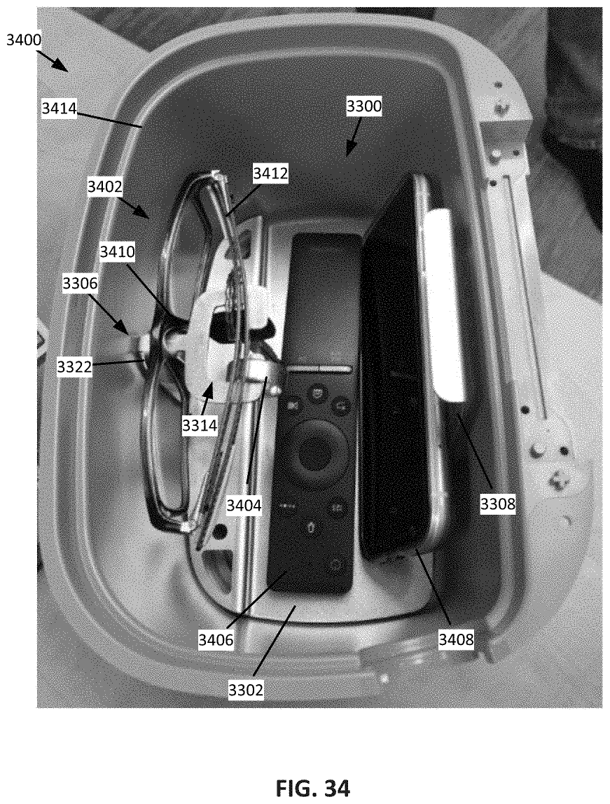

FIG. 34 shows a perspective top view of a portion of a sanitization compartment 3400, which may be an example of the sanitization compartment 104 of FIG. 1, wherein the sanitization accessory 3300 of FIG. 33 is disposed therein. As shown, the accessory rack 3306 is supporting a first article to be sanitized 3402 and a second article to be sanitized 3404, the platform 3302 is supporting a third article to be sanitized 3406, and the accessory spacer 3308 is supporting a fourth article to be sanitized 3408. As shown, the first article to be sanitized 3402 can be eyewear, the second article to be sanitized 3404 can be a ring, the third article to be sanitized 3406 can be a remote control (e.g., for controlling a television set), and the fourth article to be sanitized 3408 can be a mobile phone.

The eyewear 3402 includes nose pieces 3410 supported by the plate arms 3322 and temple (or ear) pieces 3412 that extend over the one or more plate openings 3314. The ring 3404 is received within one of the one or more plate openings 3314 and supported by the accessory rack 3306 in a suspended position relative to the platform 3302. The remote control 3406 extends along the platform 3302 and is supported thereby. The mobile phone 3408 is supported by the accessory spacer 3308 such that the mobile phone 3408 is spaced apart from one or more sidewalls 3414 of the sanitization compartment 3400.

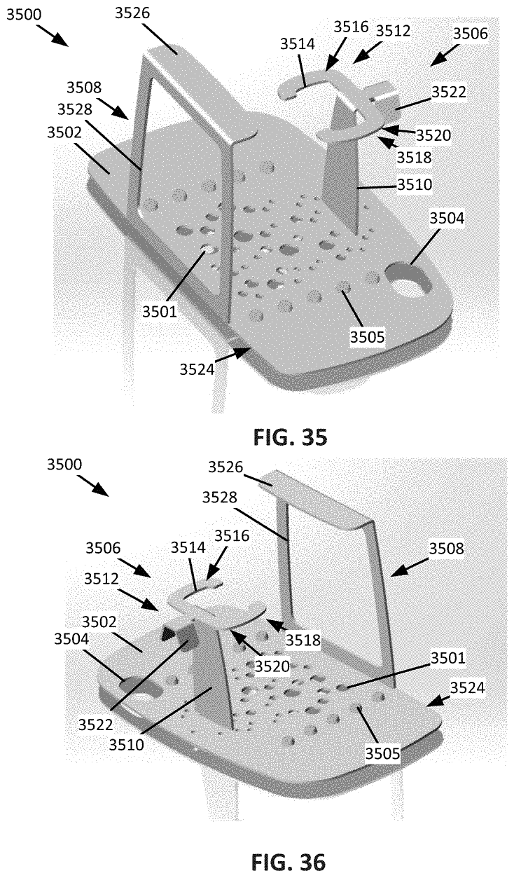

FIGS. 35 and 36 show a perspective view of a sanitization accessory 3500. The sanitization accessory 3500 includes a platform 3502, an accessory rack 3506, and an accessory spacer 3508. The sanitization accessory 3500 may be made of, for example, one or more of a metal alloy (e.g., a stainless steel alloy or an aluminum alloy), a plastic, and/or any other material.

The platform 3502 may include one or more platform openings 3501 extending therethrough. The one or more platform openings 3501 may be configured to allow ozone to pass therethrough. In some instances, at least one of the one or more platform openings 3501 may be configured to receive a standoff. The standoff may be configured to space the platform 3502 apart from a base of a sanitization compartment of a sanitization device (e.g., the sanitization compartment 104 of the sanitization device 100 of FIG. 1). The standoff may be made of a plastic, a rubber, and/or any other material. The standoff may be configured to resist a sliding movement of the platform 3502 relative to the sanitization compartment. For example, the standoff may include rubber, wherein the rubber is configured to directly engage with a portion of the sanitization compartment. Additionally, or alternatively, the standoff may extend from a corresponding platform opening 3501 and in a direction away from the base of the sanitization compartment such that an article to be sanitized engages the standoff. For example, the standoff may be configured to engage the article to be sanitized such that sliding movement of the article to be sanitized, relative to the platform 3502, is resisted as a result of the engagement. In some instances, for example, an article to be sanitized that is supported by the accessory spacer 3508 may be supported in an at least partially upright position as a result of an engagement of the article to be sanitized with the accessory spacer 3508 and at least one standoff. In some instances, and by way of further example, an article to be sanitized may be at least partially supported by one or more standoffs such that at least a portion of the article to be sanitized is spaced apart from the platform 3502. As shown, the platform 3502 may include a plurality of platform openings 3501, wherein at least one platform opening 3501 has a width (e.g., diameter) that measures differently from that of at least one other platform opening 3501.

In some instances, the platform 3502 may include a connector opening 3504 through which a connector (e.g., the connector 218 of FIG. 2) may extend. For example, the connector can be configured such that when extending through the connector opening 3504, the connector urges ozone over the platform 3502. In some instances, the platform 3502 may further include one or more protrusions 3505 extending therefrom. The protrusions 3505 may be one example of a standoff. The protrusions 3505 may be optically transparent such that light may pass therethrough. Additionally, or alternatively, the one or more protrusions 3505 may be photoluminescent. In some instances, the one or more protrusions 3505 may be configured to be illuminated by an illumination source (e.g., an LED or an incandescent bulb). For example, the one or more protrusions 3505 may include an illumination source.

The accessory rack 3506 includes a rack support 3510 extending from the platform 3502 and an article plate 3512 extending from the rack support 3510. The article plate 3512 may include one or more plate openings 3514 defined within the article plate 3512. The one or more plate openings 3514 may extend from a supporting surface 3516 and through a platform facing surface 3518 of the article plate 3512. At least one of the one or more plate openings 3514 may extend from the supporting surface 3516 through the platform facing surface 3518 and through a lateral surface 3520, wherein the lateral surface 3520 extends between the supporting surface 3516 and the platform facing surface 3518. When at least one of the one or more plate openings 3514 extends through the lateral surface 3520, a portion of an article to be sanitized (e.g., a ring, a keyring, a bracelet, watch, or a necklace) can be received within the plate opening 3514 and supported by the article plate 3512 in a suspended orientation.

The article plate 3512 may further include one or more plate arms 3522 that extend away from the article plate 3512 and in a direction of the platform 3502. In some instances, the article plate 3512 may be configured to support eyewear at a position spaced apart from the platform 3502. For example, the article plate 3512 may include a plurality of plate arms 3522, wherein the plate arms 3522 are configured to support one or more nose pieces corresponding to eyewear positioned on the article plate 3512. In this example, the temple portion (or ear pieces) of the eyewear may extend over the one or more plate openings 3514.

The accessory spacer 3508 extends from the platform 3502 and may extend along a perimeter 3524 of the platform 3502. The accessory spacer 3508 is configured to space an article to be sanitized (e.g., a mobile phone, a tablet computer, a card such as credit card, or a book) apart from the perimeter 3524. As such, when the sanitization accessory 3500 is received within a sanitization compartment of a sanitization device (e.g., the sanitization compartment 104 of the sanitization device 100 of FIG. 1), the article to be sanitized is spaced part from a sidewall of the sanitization compartment. The accessory spacer 3508 may further include a handle 3526 and one or more spacer openings 3528. The handle 3526 may facilitate removal of the sanitization accessory 3500 from a sanitization compartment of a sanitization device. The one or more spacer openings 3528 may be configured such that ozone can pass therethrough.

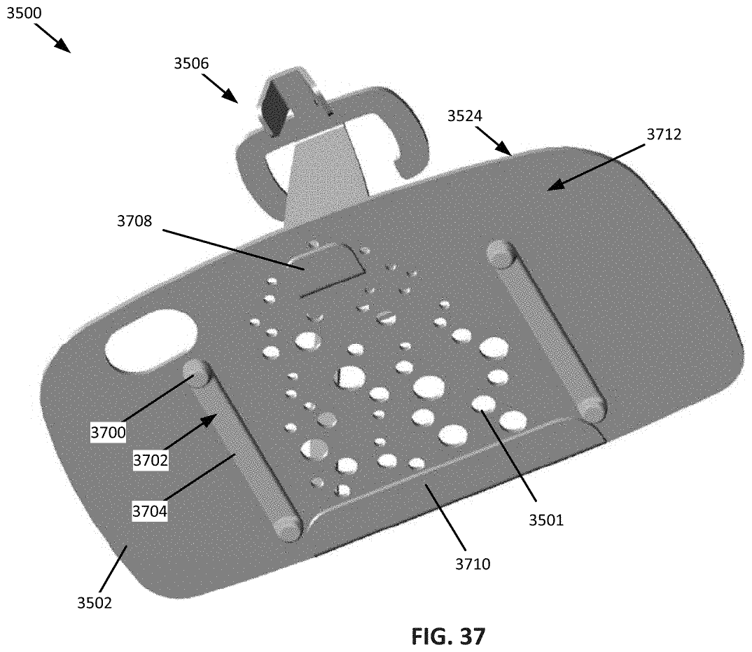

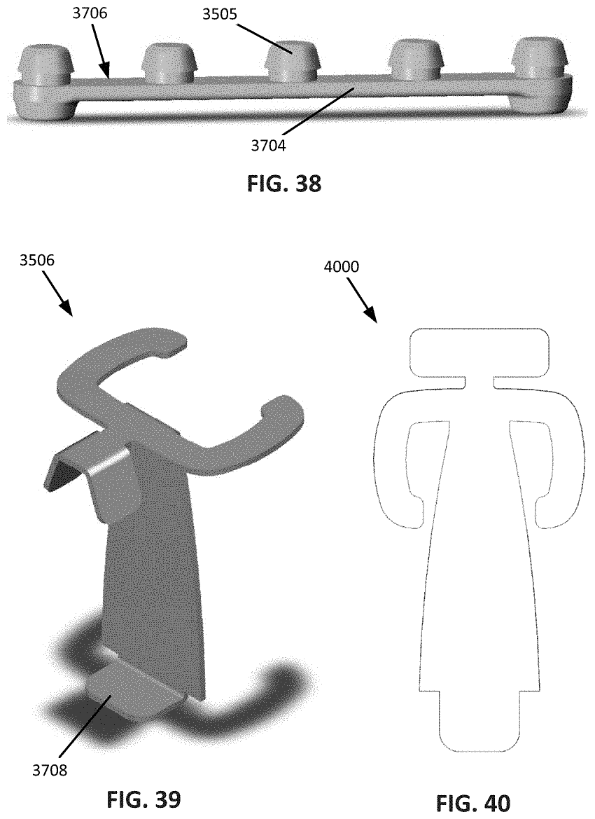

FIG. 37 shows a bottom perspective view of the sanitization accessory 3500. As shown, the sanitization accessory 3500 may include a plurality of accessory feet 3700, wherein the accessory feet 3700 may be another example of a standoff. The plurality of accessory feet 3700 extend from a bottom surface 3702 of an accessory substrate 3704. The accessory substrate 3704 may be coupled to the platform 3502 using one or more platform openings 3501. For example, the one or more protrusions 3505 may extend from a top surface 3706 (see, e.g., FIG. 38) of the accessory substrate 3704 and through respective platform openings 3501, coupling the accessory substrate 3704 to the platform 3502. As such, in some instances, the protrusions 3505 may be at least partially compressible such that the protrusions 3505 are compressed when being inserted into the platform openings 3501 and expand when fully inserted into the platform openings 3501 such that the accessory substrate 3704 is coupled to the platform 3502.

As also shown in FIG. 37, the accessory rack 3506 includes a rack bracket 3708 and the accessory spacer 3508 includes a spacer bracket 3710. The rack bracket 3708 and the spacer bracket 3710 are configured to couple the accessory rack 3506 and the accessory spacer 3508 to the platform 3502. For example, the rack bracket 3708 may be configured to extend through a bracket opening defined in the platform 3502 and the spacer bracket 3710 may be configured to extend around the perimeter 3524 of the platform 3502 and couple to a bottom surface 3712 of the platform 3502. In some instances, the accessory rack 3506 and/or the accessory spacer 3508 may be removably coupled to the platform 3502.

FIG. 39 shows an example of the accessory rack 3506 removed from the platform 3502. As shown, the accessory rack 3506 may be formed of a single material. For example, the accessory rack 3506 may be formed by folding a single sheet of material (e.g., a sheet made of a metal alloy) into the shape of the accessory rack 3506. FIG. 40 shows an example of a cutout 4000 capable of being bent into the shape of the accessory rack 3506 shown in FIG. 39.

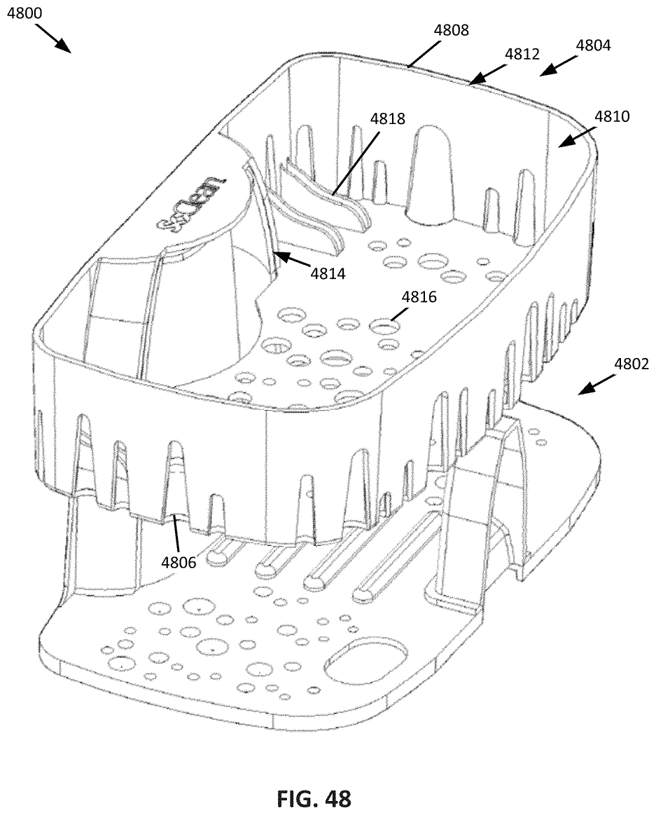

FIG. 48 shows a perspective view of a sanitization accessory 4800 having a first component 4802 and a second component 4804. The second component 4804 is supported on the first component 4802 such that at least a portion of the first component 4802 is spaced apart from at least a portion of the second component 4804. For example, the second component 4804 may be removably coupled to the first component 4802.

As shown, the second component 4804 includes a first sidewall 4806 and at least one second sidewall 4808 extending from the first sidewall 4806. The first and second sidewalls 4806 and 4808 collectively define an accessory cavity 4810 having at least one open end 4812. The at least one open end 4812 can be opposite the first sidewall 4806. As such, the second component 4804 may generally be described as being a basket.

The first and/or second sidewalls 4806 and/or 4808 may collectively define a receptacle 4814 configured to receive at least a portion of the first component 4802 such that the second component 4804 is supported by the first component 4802. In some instances, at least a portion of the receptacle 4814 may define a handle for separating the second component 4804 from the first component 4802.

The first and/or second sidewalls 4806 and/or 4808 may include one or more second component openings 4816. The one or more second component openings 4816 are configured such that ozone may pass therethrough. In some instances, the second component 4804 may include one or more supports 4818. The one or more supports 4818 may extend from one or more of the first and/or second sidewalls 4806 and/or 4808. The one or more supports 4818 can be configured to support one or more articles to be sanitized (e.g., cards such as credit cards, wallets, and/or any other article to be sanitized).

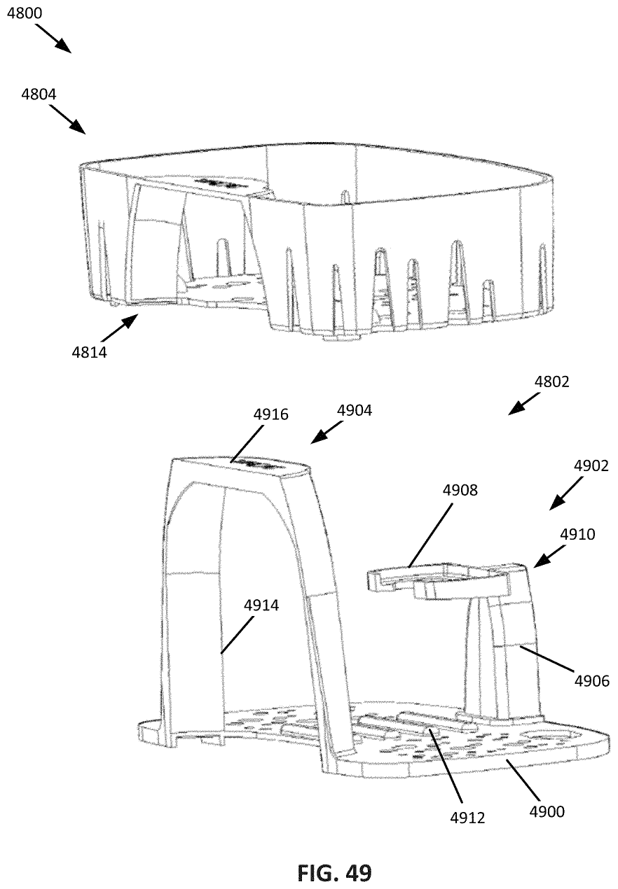

FIG. 49 shows an exploded view of the sanitization accessory 4800, wherein the first and second components 4802 and 4804 are separated. As shown, the first component 4802 includes a platform 4900, an accessory rack 4902, and an accessory spacer 4904. The accessory rack 4902 and the accessory spacer 4904 extend from the platform 4900. At least a portion of the accessory spacer 4904 is configured to be received within the receptacle 4814 of the second component 4804. As such, the receptacle 4814 and the accessory spacer 4904 may generally be described as being configured to cooperate to support the second component 4804, wherein at least a portion of the second component 4804 is spaced apart from the first component 4802. For example, the receptacle 4814 and the accessory spacer 4904 may be configured such that the second component 4804 is supported in a cantilevered configuration. By way of further example, the accessory rack 4902 may also engage at least a portion of the second component 4804, providing additional support for second component 4804.

The accessory rack 4902 includes a rack support 4906 and an article plate 4908. The rack support 4906 extends from the platform 4900 (e.g., in a vertical direction) and the article plate 4908 extends from a distal end region 4910 of the rack support 4906 (e.g., in a horizontal direction). For example, the article plate 4908 may extend in a direction away from the rack support 4906 such that at least a portion of the article plate 4908 extends over at least a portion of the platform 4900. The article plate 4908 may be configured to support an article to be sanitized (e.g., a watch, a bracelet, a necklace, a ring, and/or any other article to be sanitized) in a position that is spaced a part from the platform 4900.

The accessory spacer 4904 is configured to support an article to be sanitized in an angled orientation (relative to the accessory spacer 4904). For example, the accessory spacer 4904 can be configured to cooperate with one or more protrusions 4912 extending from the platform 4900 to support an article to be sanitized in an angled orientation. As shown, the one or more protrusions 4912 may take the form of elongate ribs that extend along a portion of the platform 4900. The protrusions 4912 may be an example of a standoff. The accessory spacer 4904 includes one or more spacer openings 4914. The one or more spacer openings 4914 are configured to allow ozone to pass therethrough. In some instances, the accessory spacer 4904 may include a first component handle 4916. The first component handle 4916 may be configured to be received within the receptacle 4814.

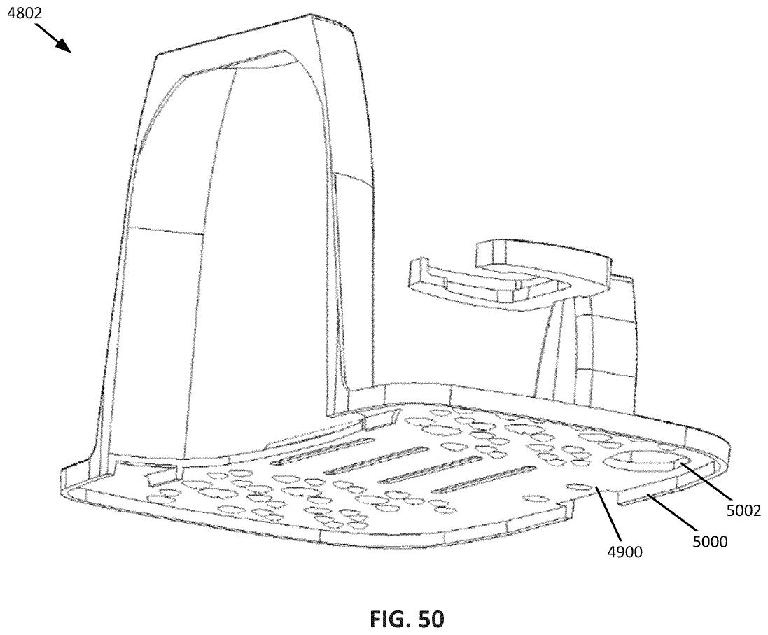

FIG. 50 shows a bottom perspective view of the first component 4802. As shown, the platform 4900 may include one or more platform sidewalls 5000 that extend from the platform 4900. When in use, the one or more platform sidewalls 5000 may extend towards a base of a sanitization compartment within which the first component 4802 is disposed. As such, the platform sidewalls 5000 may generally be described as being configured to space at least a portion of the platform 4900 apart from the base of the sanitization compartment. As also shown, the platform 4900 may include a connector opening 5002 through which a connector (e.g., the connector 218 of FIG. 2) may extend.

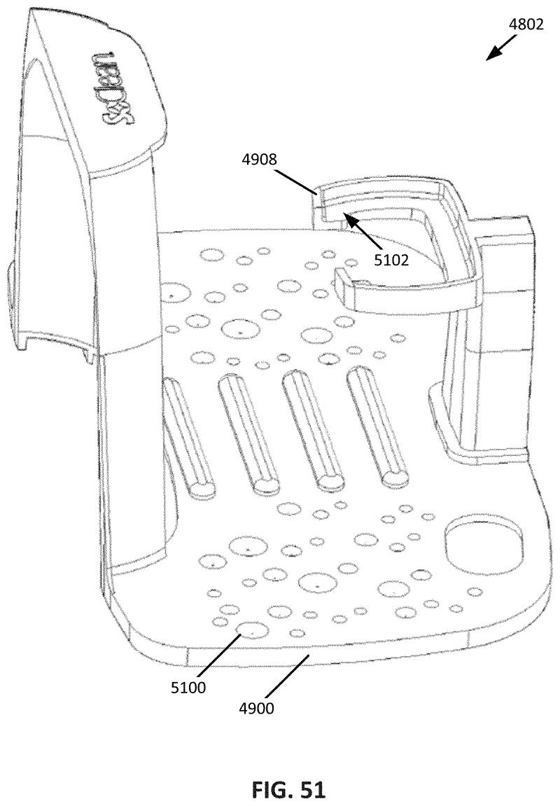

FIG. 51 shows a top perspective view of the first component 4802. As shown, the platform 4900 includes one or more platform recesses 5100 that are defined within the platform 4900. The platform recesses 5100 are shown as having a semi-spherical shape; however, other shapes are possible. The platform recesses 5100 may not extend through the platform 4900. As also shown, the article plate 4908 may include a stepped region 5102. In some instances, the stepped region 5102 may be configured to receive and support an adapter (e.g., an adapter configured to couple to an article to be sanitized).

The sanitization accessories disclosed herein may generally be described as being configured to support one or more articles to be sanitized. In some instances, the sanitization accessories may also be generally described as being configured to encourage ozone to flow around and be incident on one or more articles supported thereon. For example, the sanitization accessories may include one or more openings through which ozone can pass through.

An example of a sanitization device, consistent with the present disclosure, may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter fluidly coupled to the ozone outlet and configured to reduce ozone passing therethrough to oxygen, the filter being downstream of the connector, and an exhaust fan fluidly coupled to the ozone outlet and configured to urge ozone through the filter, the exhaust fan being configured to urge ozone through the filter while the ozone generator is generating ozone.

In some instances, the connector may be vertically spaced apart from the ozone outlet by a vertical separation distance. In some instances, the vertical separation distance may be maximized. In some instances, the connector may be horizontally spaced apart from the ozone outlet by a horizontal separation distance. In some instances, the horizontal separation distance may be maximized. In some instances, the filter and the exhaust fan may be downstream of the ozone outlet. In some instances, the exhaust fan may be downstream of the filter. In some instances, the connector may include a first segment and a second segment, the second segment extending transverse to the first segment. In some instances, the first segment may extend perpendicular to the second segment. In some instances, the sanitization compartment may include a base defining a connector opening configured to threadably receive the connector. In some instances, sanitization device may further include a seal, wherein the connector includes a flange and the seal is disposed between the flange and the base. In some instances, the base may include one or more projections.

Another example of a sanitization device, consistent with the present disclosure, may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter fluidly coupled to the ozone outlet and configured to reduce ozone passing therethrough to oxygen, the filter being downstream of the connector, an exhaust fan fluidly coupled to the ozone outlet, and a controller communicatively coupled to the exhaust fan and configured to cause the exhaust fan to urge ozone through the filter when the ozone generator is generating ozone.

In some instances, the exhaust fan may be configured to transition between an exhaust fan off state, an exhaust fan distribution state, and an exhaust fan exhaust state, a rotational speed of the exhaust fan in the exhaust fan exhaust state may measure differently from a rotational speed of the exhaust fan in the exhaust fan distribution state. In some instances, the controller may be configured to cause the exhaust fan to be in the exhaust fan distribution state when the ozone generator is generating ozone. In some instances, the controller may be configured to cause the exhaust fan to transition from the exhaust fan distribution state to the exhaust fan exhaust state when the ozone generator discontinues generating ozone. In some instances, the controller may be configured to cause the exhaust fan to transition from the exhaust fan exhaust state to the exhaust fan off state after the ozone generator has discontinued generating ozone for an exhaust time period. In some instances, the connector may be vertically spaced apart from the ozone outlet by a vertical separation distance and the connector may be horizontally spaced apart from the ozone outlet by a horizontal separation distance. In some instances, the vertical separation distance may be maximized and the horizontal separation distance may be maximized. In some instances, the connector may include a first segment and a second segment, the second segment extending transverse to the first segment.

Another example of a sanitization device, consistent with the present disclosure, may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter access door transitionable between an open position and a closed position, the filter access door including a filter holder, a filter configured to be disposed within the filter holder, the filter being fluidly coupled to the ozone outlet when the filter access door is in the closed position and the filter being configured to reduce ozone passing therethrough to oxygen, the filter being downstream of the connector, and an exhaust fan fluidly coupled to the ozone outlet and configured to urge ozone through the filter.

In some instances, the connector may be vertically spaced apart from the ozone outlet by a vertical separation distance. In some instances, the connector may be horizontally spaced apart from the ozone outlet by a horizontal separation distance. In some instances, one or more of the vertical separation distance or the horizontal separation distance may be maximized. In some instances, the filter access door may include a fluid guide configured to urge a fluid exhausted from the filter toward the exhaust fan. In some instances, the filter and the exhaust fan may be downstream of the ozone outlet. In some instances, the exhaust fan may be downstream of the filter. In some instances, the connector may include a first segment and a second segment, the second segment extending transverse to the first segment. In some instances, the first segment may extend perpendicular to the second segment. In some instances, the sanitization compartment may include a base defining a connector opening configured to threadably receive the connector. In some instances, the sanitization device may further include a seal, wherein the connector includes a flange and the seal is disposed between the flange and the base. In some instances, the exhaust fan may be coupled to the filter access door.

Another example of a sanitization device, consistent with the present disclosure, may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter access door transitionable between an open position and a closed position, the filter access door including a filter holder, a filter configured to be disposed within the filter holder, the filter being fluidly coupled to the ozone outlet when the filter access door is in the closed position and the filter being configured to reduce ozone passing therethrough to oxygen, the filter being downstream of the connector, an exhaust fan fluidly coupled to the ozone outlet, and a controller communicatively coupled to the exhaust fan and configured to cause the exhaust fan to urge ozone through the filter.

In some instances, the exhaust fan may be configured to transition between an exhaust fan off state, an exhaust fan distribution state, and an exhaust fan exhaust state, a rotational speed of the exhaust fan in the exhaust fan exhaust state measures differently from a rotational speed of the exhaust fan in the exhaust fan distribution state. In some instances, the controller may be configured to cause the exhaust fan to be in the exhaust fan distribution state when the ozone generator is generating ozone. In some instances, the controller may be configured to cause the exhaust fan to transition from the exhaust fan distribution state to the exhaust fan exhaust state when the ozone generator discontinues generating ozone. In some instances, the controller may be configured to cause the exhaust fan to transition from the exhaust fan exhaust state to the exhaust fan off state after the ozone generator has discontinued generating ozone for an exhaust time period. In some instances, the connector may be vertically spaced apart from the ozone outlet by a vertical separation distance and the connector is horizontally spaced apart from the ozone outlet by a horizontal separation distance. In some instances, the filter access door may include a fluid guide configured to urge a fluid exhausted from the filter toward the exhaust fan. In some instances, the connector may include a first segment and a second segment, the second segment extending transverse to the first segment.

An example of a sanitization system, consistent with the present disclosure, may include a sanitization device and a sanitization accessory. The sanitization device may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter fluidly coupled to the ozone outlet and configured to reduce ozone passing therethrough to oxygen, the filter being downstream of the connector, and an exhaust fan fluidly coupled to the ozone outlet and configured to urge ozone through the filter. The sanitization accessory may be disposed within the sanitization compartment and configured to support an article to be sanitized.

In some instances, the exhaust fan may be configured to urge ozone through the filter while the ozone generator is generating ozone. In some instances, the sanitization accessory may include a platform having one or more platform openings. In some instances, the sanitization accessory may include one or more sanitization racks extending from the platform. In some instances, the one or more sanitization racks may be removably coupled to a corresponding platform opening. In some instances, the one or more sanitization racks may include one or more rack arms. In some instances, the one or more rack arms may include one or more arm recesses. In some instances, the sanitization rack may include an article plate. In some instances, the sanitization accessory may include one or more accessory risers extending from the platform. In some instances, the sanitization accessory may include a plurality of sanitization risers extending from the platform, the sanitization risers being spaced apart from each other. In some instances, the sanitization risers may extend parallel to each other. In some instances, the sanitization accessory may include one or more accessory spacers extending from the platform. In some instances, the one or more accessory spacers may have a frustoconical shape.

Another example of a sanitization device, consistent with the present disclosure, may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter access door transitionable between an open position and a closed position, the filter access door including a filter holder configured to receive a filter such that the filter fluidly couples to the ozone outlet when the filter access door is in the closed position, the filter is configured to reduce ozone passing therethrough to oxygen and the filter is downstream of the connector, and an exhaust fan fluidly coupled to the ozone outlet and configured to urge ozone through the filter.

In some instances, the connector may be vertically spaced apart from the ozone outlet by a vertical separation distance. In some instances, the connector may be horizontally spaced apart from the ozone outlet by a horizontal separation distance. In some instances, one or more of the vertical separation distance or the horizontal separation distance may be maximized. In some instances, the filter access door may include a fluid guide configured to urge a fluid exhausted from the filter toward the exhaust fan. In some instances, the filter and the exhaust fan may be downstream of the ozone outlet. In some instances, the exhaust fan may be downstream of the filter. In some instances, the connector may include a first segment and a second segment, the second segment extending transverse to the first segment. In some instances, the first segment may extend perpendicular to the second segment. In some instances, the sanitization compartment may include a base defining a connector opening configured to threadably receive the connector. In some instances, the sanitization device may further include a seal, wherein the connector includes a flange and the seal is disposed between the flange and the base. In some instances, the exhaust fan may be coupled to the filter access door.

Another example of a sanitization device, consistent with the present disclosure, may include a sanitization compartment including an ozone outlet, a connector defining a fluid pathway that is fluidly coupled to the sanitization compartment, an ozone operating system having an ozone generator configured to generate ozone and a distribution fan configured to urge ozone generated by the ozone generator through the fluid pathway of the connector and into the sanitization compartment, a filter access door transitionable between an open position and a closed position, the filter access door including a filter holder, a filter disposed within the filter holder such that the filter fluidly couples to the ozone outlet when the filter access door is in the closed position, the filter is configured to reduce ozone passing therethrough to oxygen and the filter is downstream of the connector, and an exhaust fan fluidly coupled to the ozone outlet and configured to urge ozone through the filter.

In some instances, the filter may include a housing and a filter medium extending within the housing. In some instances, the housing may include a first shell configured to couple to a second shell, a cavity for receiving the filter medium extends between the first shell and the second shell. In some instances, the filter medium may have a filtration efficiency of at least 90%. In some instances, the filter medium may be an activated carbon filter having a carbon density of 1600 grams per cubic meter. In some instances, the filter medium may be an activated carbon filter having 30 pores-per-inch. In some instances, the exhaust fan may be coupled to the filter access door. In some instances, the filter access door may include a fluid guide configured to urge a fluid exhausted from the filter toward the exhaust fan. In some instances, the filter and the exhaust fan may be downstream of the ozone outlet. In some instances, the connector may include a first segment and a second segment, the second segment extending transverse to the first segment.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the claims.

What is claimed is:

1. A sanitization device comprising:
a sanitization compartment including an ozone outlet;
a connector defining a fluid pathway that is fluidly coupled to the sanitization compartment;
an ozone operating system having an ozone generator configured to generate ozone and a distribution fan configured to urge ozone generated by the ozone generator through the fluid pathway of the connector and into the sanitization compartment;
a filter access door transitionable between an open position and a closed position, the filter access door including a filter holder configured to receive a filter such that the filter fluidly couples to the ozone outlet when the filter access door is in the closed position, the filter is configured to reduce ozone passing therethrough to oxygen and the filter is downstream of the connector; and
an exhaust fan fluidly coupled to the ozone outlet and configured to urge ozone through the filter.

2. The sanitization device of claim 1, wherein the connector is vertically spaced apart from the ozone outlet by a vertical separation distance.

3. The sanitization device of claim 2, wherein the connector is horizontally spaced apart from the ozone outlet by a horizontal separation distance.

4. The sanitization device of claim 3, wherein one or more of the vertical separation distance or the horizontal separation distance is maximized.

5. The sanitization device of claim 1, wherein the filter access door includes a fluid guide configured to urge a fluid exhausted from the filter toward the exhaust fan.

6. The sanitization device of claim 1, wherein the filter and the exhaust fan are downstream of the ozone outlet.

7. The sanitization device of claim 6, wherein the exhaust fan is downstream of the filter.

8. The sanitization device of claim 1, wherein the connector includes a first segment and a second segment, the second segment extending transverse to the first segment.

9. The sanitization device of claim 8, wherein the first segment extends perpendicular to the second segment.

10. The sanitization device of claim 1, wherein the sanitization compartment includes a base defining a connector opening configured to threadably receive the connector.

11. The sanitization device of claim 10 further comprising a seal, wherein the connector includes a flange and the seal is disposed between the flange and the base.

12. The sanitization device of claim 1, wherein the exhaust fan is coupled to the filter access door.

13. A sanitization device comprising:
a sanitization compartment including an ozone outlet;
a connector defining a fluid pathway that is fluidly coupled to the sanitization compartment;
an ozone operating system having an ozone generator configured to generate ozone and a distribution fan configured to urge ozone generated by the ozone generator through the fluid pathway of the connector and into the sanitization compartment;
a filter access door transitionable between an open position and a closed position, the filter access door including a filter holder;
a filter configured to be disposed within the filter holder, the filter being fluidly coupled to the ozone outlet when the filter access door is in the closed position and the filter being configured to reduce ozone passing therethrough to oxygen, the filter being downstream of the connector;
an exhaust fan fluidly coupled to the ozone outlet; and
a controller communicatively coupled to the exhaust fan and configured to cause the exhaust fan to urge ozone through the filter.

14. The sanitization device of claim 13, wherein the exhaust fan is configured to transition between an exhaust fan off state, an exhaust fan distribution state, and an exhaust fan exhaust state, a rotational speed of the exhaust fan in the exhaust fan exhaust state measures differently from a rotational speed of the exhaust fan in the exhaust fan distribution state.

15. The sanitization device of claim 14, wherein the controller is configured to cause the exhaust fan to be in the exhaust fan distribution state when the ozone generator is generating ozone.

16. The sanitization device of claim 14, wherein the controller is configured to cause the exhaust fan to transition from the exhaust fan distribution state to the exhaust fan exhaust state when the ozone generator discontinues generating ozone.

17. The sanitization device of claim 14, wherein the controller is configured to cause the exhaust fan to transition from the exhaust fan exhaust state to the exhaust fan off state after the ozone generator has discontinued generating ozone for an exhaust time period.

18. The sanitization device of claim 13, wherein the connector is vertically spaced apart from the ozone outlet by a vertical separation distance and the connector is horizontally spaced apart from the ozone outlet by a horizontal separation distance.

19. The sanitization device of claim 13, wherein the filter access door includes a fluid guide configured to urge a fluid exhausted from the filter toward the exhaust fan.

20. The sanitization device of claim 13, wherein the connector includes a first segment and a second segment, the second segment extending transverse to the first segment.

21. A sanitization device comprising:
a sanitization compartment including an ozone outlet;
a connector defining a fluid pathway that is fluidly coupled to the sanitization compartment;
an ozone operating system having an ozone generator configured to generate ozone and a distribution fan configured to urge ozone generated by the ozone generator through the fluid pathway of the connector and into the sanitization compartment;
a filter access door transitionable between an open position and a closed position, the filter access door including a filter holder;
a filter disposed within the filter holder such that the filter fluidly couples to the ozone outlet when the filter access door is in the closed position, the filter is configured to reduce ozone passing therethrough to oxygen and the filter is downstream of the connector; and
an exhaust fan fluidly coupled to the ozone outlet and configured to urge ozone through the filter.

22. The sanitization device of claim 21, wherein the filter includes a housing and a filter medium extending within the housing.

23. The sanitization device of claim 22, wherein the housing includes a first shell configured to couple to a second shell, a cavity for receiving the filter medium extends between the first shell and the second shell.

24. The sanitization device of claim 22, wherein the filter medium has a filtration efficiency of at least 90%.

25. The sanitization device of claim 22, wherein the filter medium is an activated carbon filter having a carbon density of 1600 grams per cubic meter.

26. The sanitization device of claim 22, wherein the filter medium is an activated carbon filter having 30 pores-per-inch.

27. The sanitization device of claim 21, wherein the exhaust fan is coupled to the filter access door.

28. The sanitization device of claim 21, wherein the filter access door includes a fluid guide configured to urge a fluid exhausted from the filter toward the exhaust fan.

29. The sanitization device of claim 21, wherein the filter and the exhaust fan are downstream of the ozone outlet.

30. The sanitization device of claim 21, wherein the connector includes a first segment and a second segment, the second segment extending transverse to the first segment.
